(19) United States
(12) Patent Application Publication
(10) Pub. No.: US 2023/0310424 A1
(43) Pub. Date: Oct. 5, 2023

(54) METHODS AND MATERIALS FOR INCREASING NICOTINAMIDE PHOSPHORIBOSYL TRANSFERASE ACTIVITY

(71) Applicant: University of Pittsburgh - Of the Commonwealth System of Higher Education, Pittsburgh, PA (US)

(72) Inventors: Beibei Chen, Pittsburgh, PA (US); Toren Finkel, Pittsburgh, PA (US); Yuan Liu, Pittsburgh, PA (US)

(21) Appl. No.: 18/022,677

(22) PCT Filed: Nov. 19, 2021
(86) PCT No.: PCT/US2021/060158

§ 371 Date: Feb. 22, 2023

Related U.S. Application Data
(60) Provisional application No. 63/116,767, filed on Nov. 20, 2020.

(51) Int. Cl. A61K 31/498; A61K 31/4985

(52) U.S. Cl. CPC A61K 31/498; A61K 31/4985

(57) ABSTRACT

This document provides compounds that can increase NAMPT activity, as well as the methods of using such compounds to treat diseases, disorders, and conditions such as traumatic nerve and/or brain injuries, chemotherapeutic-induced or diabetic neuropathies, and neurodegenerative diseases.

### CLAIM OF PRIORITY

This application claims priority to U.S. Patent Application Ser. No. 63/116,767, filed on Nov. 20, 2020, the entire contents of which are hereby incorporated by reference.

### FEDERALLY SPONSORED RESEARCH AND DEVELOPMENT

This invention was made with government support under grant number HL139860 awarded by National Institutes of Health (NIH). The government has certain rights in the invention.

### TECHNICAL FIELD

This document relates to methods and materials for activating nicotinamide phosphoribosyltransferase (NAMPT) activity. For example, this document provides compounds (e.g., organic compounds) having the ability to increase NAMPT activity within cells and/or within a mammal, formulations containing one or more compounds having the ability to increase NAMPT activity within cells and/or within a mammal, methods for making one or more compounds having the ability to increase NAMPT activity within cells and/or within a mammal, methods for increasing NAMPT activity within cells and/or within a mammal, and methods for treating mammals (e.g., humans) having a condition responsive to increased NAMPT activity.

### BACKGROUND

Intracellularily, the enzyme NAMPT is a rate-limiting enzyme of the nicotinamide adenine dinucleotide (NAD⁺) salvage pathway that converts nicotinamide to nicotinamide mononucleotide (NMN) and forms most of the NAD⁺ found in mammals (Revollo et al., Current Opinion in Gastroenterology, 23(2):164-70 (2007)). Intracellular NAMPT also catalyzes the synthesis of NMN from phosphoribosyl pyrophosphate (PRPP) in the presence of ATP (Galli et al., Frontiers in Pharmacology, 11:656 (2020)). In some cases, extracellular NAMPT can act as a cytokine involved in promoting B cell maturation and inhibiting neutrophil apoptosis (Samal et al., Mol. Cell. Biol., 14(2):1431-7 (1994)).

### SUMMARY

This document provides methods and materials for increasing NAMPT activity. For example, this document provides compounds (e.g., organic compounds) having the ability to increase NAMPT activity within cells and/or within a mammal, formulations containing one or more compounds having the ability to increase NAMPT activity within cells and/or within a mammal, methods for making one or more compounds having the ability to increase NAMPT activity within cells and/or within a mammal, methods for making formulations containing one or more compounds having the ability to increase NAMPT activity within cells and/or within a mammal, methods for increasing NAMPT activity within cells and/or within a mammal, and methods for treating mammals (e.g., humans) having a condition responsive to an increase of NAMPT activity. Suitable examples of conditions responsive to an increase of NAMPT activity within cells and/or within the mammal include, without limitation, traumatic nerve injuries, traumatic brain injuries, chemotherapeutic-induced neuropathies, diabetic neuropathies, and neurodegenerative diseases. The methods and materials for increasing NAMPT activity as described herein also can be used to increase NAD⁺ levels to slow cognitive decline, vision loss, and/or hearing loss and/or to prolong lifespan (see, e.g., Rajman et al., Cell Metabolism, 27:529-547 (2018)). In some cases, methods and materials for increasing NAMPT activity as described herein can be used to increase NAD⁺ levels to treat hepatosteatosis, insulin resistance syndromes and/or their associated manifestations, obesity, sarcopenia, disorders of inflammation, autoimmune conditions, skin aging, cardiovascular diseases (e.g., heart failure), acute and/or chronic kidney injury, and/or infertility. In some cases, methods and materials for increasing NAMPT activity as described herein can be used to increase NAD⁺ levels to reduce the likelihood of developing skin cancer.

As described herein, the methods and materials provided herein can be used to increase NAMPT activity within cells in vitro, in vivo, or ex vivo. In some cases, the compounds provided herein can be used to treat mammals (e.g., humans) having a disease, disorder, or condition associated with reduced levels of an NAMPT polypeptide within cells and/or within a mammal and/or associated with reduced levels of NAD⁺ formation within cells and/or within the mammal. In some cases, one or more compounds provided herein can be used to treat mammals (e.g., humans) having a disease, disorder, or condition that is responsive to increasing NAMPT activity.

In one general aspect, the present disclosure provides a method for increasing NAMPT activity within a mammal, wherein said method comprises administering, to said mammal, an effective amount of a compound of Formula (I):

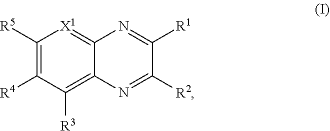

or a pharmaceutically acceptable salt thereof, wherein X¹, R¹, R², R³, R⁴, and R⁵ are as described herein.

In another general aspect, the present disclosure provides a pharmaceutical composition comprising a compound of Formula (I), or a pharmaceutically acceptable salt thereof, and a pharmaceutically acceptable carrier.

In yet another general aspect, the present disclosure provides a method of treating a mammal having a disease, disorder, or condition responsive to increasing NAMPT activity within the mammal, wherein said method comprises administering, to said mammal, any one of the compounds described herein as having the ability to increase NAMPT activity, or a pharmaceutical composition comprising same. In some embodiments, the mammal is human. In some embodiments, the method comprises treating a mammal having a traumatic nerve and/or brain injury. In some embodiments, the method comprises treating a mammal having neurodegenerative disease. In some embodiments, the method comprises treating a mammal having hepatosteatosis or liver dysfunction or a mammal that progressed to nonalcoholic steatohepatitis (NASH). In some embodiments, the method comprises treating a mammal having heart failure caused by ischemia, a genetic predisposition, or an environmental exposure. In some embodiments, the method comprises treating a mammal having hepatosteatosis, an insulin resistance syndrome and/or an associated manifestation, obesity, sarcopenia, a disorder of inflammation, an autoimmune condition, a cardiovascular disease, heart failure, an acute and/or chronic kidney injury, or infertility.

In yet another general aspect, the present disclosure provides a method for increasing NAMPT activity within a mammal, wherein said method comprises administering, to said mammal, any one of the compounds described herein as having the ability to increase NAMPT activity or a pharmaceutical composition comprising same.

In general, one aspect of this document features a method for increasing NAMPT activity within a mammal. The method comprises (or consists essentially of or consists of) administering, to the mammal, an effective amount of a compound of Formula (I):

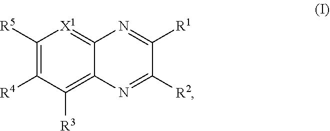

or a pharmaceutically acceptable salt thereof, wherein:
X¹ is selected from N and CR⁶;
R³, R⁴, R⁵, and R⁶ are each independently selected from H, Cy¹, halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, C(NR^{e1})NR^{c1}R^{d1}, C(NR^{e1})NR^{c1}OR^{d1}, NR^{c1}C(O)NR^{c1}R^{d1}, NR^{c1}C(S)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}; wherein the C₁₋₆ alkyl, C₂₋₆ alkenyl, and C₂₋₆ alkynyl are each optionally substituted with 1, 2, or 3 substituents independently selected from R⁷;
each R⁷ is independently selected from Cy¹, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
each Cy¹ is independently selected from C₆₋₁₀ aryl, C₃₋₁₀ cycloalkyl, 5-10 membered heteroaryl, and 4-10 membered heterocycloalkyl, each of which is optionally substituted with 1, 2, 3, 4, or 5 substituents independently selected from R^{Cy1};
each R^{Cy1} is independently selected from halo, CN, NO₂, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 5-10 membered heteroaryl 4-10 membered heterocycloalkyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}, wherein the C₁₋₆ alkyl, C₂₋₆ alkenyl, C₂₋₆ alkynyl, C₃₋₁₀ cycloalkyl, C₆₋₁₀ aryl, 5-10 membered heteroaryl, and 4-10 membered heterocycloalkyl are each optionally substituted with 1, 2, or 3 substituents independently selected from R⁸;
each R⁸ is independently selected from C₁₋₆ alkyl, C₂₋₆ alkenyl, C₂₋₆ alkynyl, C₃₋₁₀ cycloalkyl, C₆₋₁₀ aryl, 5-10 membered heteroaryl, 4-10 membered heterocycloalkyl, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}, wherein the C₁₋₆ alkyl, C₂₋₆ alkenyl, C₂₋₆ alkynyl, C₃₋₁₀ cycloalkyl, C₆₋₁₀ aryl, 5-10 membered heteroaryl, and 4-10 membered heterocycloalkyl are each optionally substituted with 1, 2, 3, 4, or 5 substituents independently selected from R^{g};
R¹ and R² are each independently selected from R⁹ and S(O)₂R⁹;
each R⁹ is independently selected from C₁₋₆ haloalkyl, C₆₋₁₀ aryl, 5-10 membered heteroaryl, and 4-10 membered heterocycloalkyl, each of which is optionally substituted with 1, 2, 3, 4, or 5 substituents independently selected from R¹⁰;
each R¹⁰ is independently selected from halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein the C₁₋₆ alkyl, C₂₋₆ alkenyl, and C₂₋₆ alkynyl are each optionally substituted with 1, 2, or 3 substituents independently selected from R¹¹;
each R¹¹ is independently selected from CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
each R^{e1} is selected from H, OR^{a1}, NR^{c1}R^{d1}, and C₁₋₄ haloalkyl;
each R^{a1}, R^{b1}, R^{c1}, and R^{d1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, 3, 4, or 5 substituents independently selected from R^{g};
or any R^{c1} and R^{d1} together with the N atom to which they are attached form a 4-7 membered heterocycloalkyl, which is optionally substituted with 1, 2, or 3 substituents independently selected from R^{g}; and
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkenylcarbonyl, C₁₋₆ alkynylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, and any C₁₋₆ alkyl, C₁₋₆ alkoxy, C₆₋₁₀ aryl, C₃₋₁₀ cycloalkyl, 5-10 membered heteroaryl, or 4-10 membered heterocycloalkyl of R^{g} is optionally substituted with 1, 2, or 3 substituents independently selected from OH, NO₂, CN, halo, C₁₋₆ alkyl, C₁₋₄ haloalkyl, C₁₋₆ alkoxy, and C₁₋₆ haloalkoxy.

In one embodiment, the compound of Formula (I) can have formula:

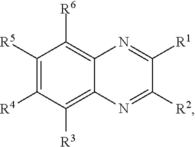

or a pharmaceutically acceptable salt thereof. In some cases, R³, R⁵, and R⁶ can be each independently selected from H, halo, C₁₋₆ alkyl, C₁₋₆ haloalkyl, OR^{a1}, C(O)OR^{a1}, and NR^{c1}R^{d1} wherein the C₁₋₆ alkyl is optionally substituted with a substituent selected from OR^{a1}, C(O)R^{b1}, C(O)NR^{c1}R^{d1}, C(O)OR^{a1}, NR^{c1}R^{d1}, S(O)₂R^{b1}, and S(O)₂NR^{c1}R^{d1}; and R⁴ can be selected from Cy¹, C(O)NR^{c1}R^{d1}, C(O)OR^{a1}, NR^{c1}R^{d1}, NR^{c1}C(O)NR^{c1}R^{d1}, NR^{c1}C(S)NR^{c1}R^{d1}, and S(O)₂NR^{c1}R^{d1}. In some cases, R³, R⁵, and R⁶ can be each independently selected from H, halo, C₁₋₆ alkyl, C₁₋₆ haloalkyl, C₁₋₆ alkoxy, C₁₋₆ haloalkoxy; and R⁴ can be selected from Cy¹, C(O)NR^{c1}R^{d1}, and C(O)OR^{a1}.

In another embodiment, the compound of Formula (I) can have formula:

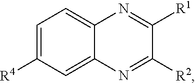

or a pharmaceutically acceptable salt thereof. The compound of Formula (I) can have formula:

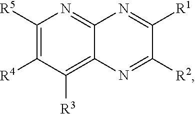

or a pharmaceutically acceptable salt thereof. In some cases, R³ and R⁵ can be each independently selected from H, halo, C₁₋₆ alkyl, C₁₋₆ haloalkyl, OR^{a1}, C(O)OR^{a1}, and NR^{c1}R^{d1} wherein the C₁₋₆ alkyl is optionally substituted with a substituent selected from OR^{a1}, C(O)R^{b1}, C(O)NR^{c1}R^{d1}, C(O)OR^{a1}, NR^{c1}R^{d1}, S(O)₂R^{b1}, and S(O)₂NR^{c1}R^{d1}; and R⁴ can be selected from Cy¹, C(O)NR^{c1}R^{d1}, C(O)OR^{a1}, NR^{c1}R^{d1}, NR^{c1}C(O)NR^{c1}R^{d1}, NR^{c1}C(S)NR^{c1}R^{d1}, and S(O)₂NR^{c1}R^{d1}, R³ and R⁵ can be each independently selected from H, halo, C₁₋₆ alkyl, C₁₋₆ haloalkyl, C₁₋₆ alkoxy, C₁₋₆ haloalkoxy; and R⁴ can be selected from Cy¹, C(O)NR^{c1}R^{d1}, and C(O)OR^{a1}.

In another embodiment, the compound of Formula (I) can have formula:

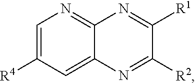

or a pharmaceutically acceptable salt thereof. R⁴ can be Cy¹. R⁴ can be C(O)NR^{c1}R^{d1}. R⁴ can be C(O)OR^{a1}.

In another embodiment, the compound of Formula (I) can have formula:

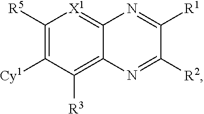

or a pharmaceutically acceptable salt thereof.

In another embodiment, The compound of Formula (I) can have formula:

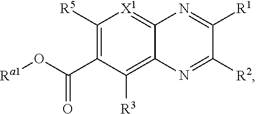

or a pharmaceutically acceptable salt thereof.

In another embodiment, the compound of Formula (I) can have formula:

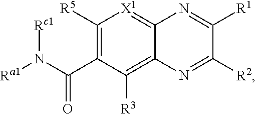

or a pharmaceutically acceptable salt thereof.

In another embodiment, the compound of Formula (I) can have formula:

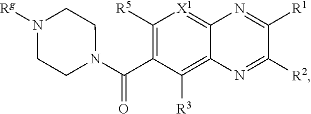

or a pharmaceutically acceptable salt thereof.

In some cases, R³, R⁵, and R⁶ can be each independently selected from H, halo, C₁₋₆ alkyl, C₁₋₆ haloalkyl, OR^{a1}, C(O)OR^{a1}, and NR^{c1}R^{d1}, wherein the C₁₋₆ alkyl is optionally substituted with a substituent selected from OR^{a1}, C(O)R^{b1}, C(O)NR^{c1}R^{d1}, C(O)OR^{a1}NR^{c1}R^{d1}, S(O)₂R^{b1}, and S(O)₂NR^{c1}R^{d1}. In some cases, R³, R⁵, and R⁶ can be each independently selected from H, halo, C₁₋₆ alkyl, C₁₋₆ haloalkyl, C₁₋₆ alkoxy, and C₁₋₆ haloalkoxy. In some cases, R³, R⁵, and R⁶ can be each H. In some cases, R^{c1} and R^{d1} can be each independently selected from H, C₁₋₆ alkyl, C₁₋₄ haloalkyl, C₂₋₆ alkenyl, and C₂₋₆ alkynyl, wherein the C₁₋₆ alkyl, C₂₋₆ alkenyl, and C₂₋₆ alkynyl are each optionally substituted with a substituent selected from OH, thio, C₁₋₆ alkoxy, C₁₋₆ thialkoxy, C₁₋₆ haloalkoxy, amino, C₁₋₆ alkylamino, and di(C₁₋₆ alkyl)amino. In some cases, R^{c1} and R^{d1} can be each independently selected from H, C₁₋₆ alkyl, and C₂₋₆ alkynyl, wherein the C₁₋₆ alkyl is optionally substituted with a substituent selected from OH, C₁₋₆ alkoxy, and di(C₁₋₆ alkyl)amino. In some cases, R^{c1} and R^{d1} together with the N atom to which they are attached can form a 4-7 membered heterocycloalkyl, which is optionally substituted with 1 or 2 substituents independently selected from R^{g}. In some cases, R^{c1} and R^{d1} together with the N atom to which they are attached can form a ring selected from morpholinyl, thiomorpholinyl, piperazinyl, piperidinyl, pyrrolidinyl, and azepanyl, each of which is optionally substituted with 1 or 2 substituents independently selected from R^{g}. In some cases, R^{c1} and R^{d1} together with the N atom to which they are attached can form a piperazinyl ring of formula:

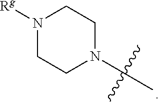

In some cases, R^{a1} can be selected from H, C₁₋₆ alkyl, C₁₋₄ haloalkyl, C₂₋₆ alkenyl, and C₂₋₆ alkynyl, wherein the C₁₋₆ alkyl, C₂₋₆ alkenyl, and C₂₋₆ alkynyl are each optionally substituted with a substituent selected from OH, thio, C₁₋₆ alkoxy, C₁₋₆ thialkoxy, C₁₋₆ haloalkoxy, amino, C₁₋₆ alkylamino, and di(C₁₋₆ alkyl)amino. In some cases, R^{a1} can be selected from H and C₁₋₆ alkyl. In some cases, Cy¹ can be 5-10 membered heteroaryl, which is optionally substituted with 1 or 2 independently selected R^{Cy1}. In some cases, Cy¹ can be selected from 1,2,4-triazolyl, tetrazolyl, oxazolyl, isoxazolyl, thiophenyl, indolyl, pyrimidinyl, pyrrolopyridinyl, benzoxadiazolyl, 1,3,4-oxadiazolyl, 1,2,3-triazolyl, 1,2,4-oxadiazolyl, thiazolyl, pyridinyl, benzoxazinyl, pyrazolyl, and indazolyl, each of which is optionally substituted with 1 or 2 substituents independently selected from R. In some cases, Cy¹ can be selected from 1,2,4-triazolyl, tetrazolyl, oxazolyl, and 1,2,4-oxadiazolyl, each of which is optionally substituted with 1 or 2 substituents independently selected from R^{Cy1}. In some cases, R^{Cy1} can be selected from C₁₋₆ alkyl, C₁₋₆ haloalkyl, C₂₋₆ alkenyl, and C₂₋₆ alkynyl, each of which is optionally substituted with 1 or 2 independently selected R⁸. In some cases, R^{Cy1} can be C₁₋₆ alkyl, optionally substituted with R⁸. In some cases, R⁸ can be selected from OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}, In some cases, R⁸ can be selected from OH, thio, C₁₋₆ alkoxy, C₁₋₆ thialkoxy, C₁₋₆ haloalkoxy, amino, C₁₋₆ alkylamino, and di(C₁₋₆ alkyl)amino. In some cases, R¹ and R² can be each independently selected from C₁₋₆ haloalkyl, C₆₋₁₀ aryl, and 5-10 membered heteroaryl, wherein the C₆₋₁₀ aryl and 5-10 membered heteroaryl are each optionally substituted with 1 or 2 independently selected from R¹⁰. In some cases, R¹ and R² can be each independently a C₆₋₁₀ aryl, optionally substituted with 1 or 2 independently selected from R¹⁰. In some cases, R¹ can be 5-10 membered heteroaryl, optionally substituted with 1 or 2 independently selected from R¹⁰; and R² can be C₆₋₁₀ aryl, optionally substituted with 1 or 2 independently selected from R¹⁰. In some cases, R¹ can be C₆₋₁₀ aryl, optionally substituted with 1 or 2 independently selected from R¹⁰; and R² can be 5-10 membered heteroaryl, optionally substituted with 1 or 2 independently selected from R¹⁰. In some cases, R¹ can be C₁₋₆ haloalkyl; and R² can be C₆₋₁₀ aryl, optionally substituted with 1 or 2 independently selected from R¹⁰. In some cases, R¹ can be C₁₋₆ haloalkyl; and R² can be 5-10 membered heteroaryl, optionally substituted with 1 or 2 independently selected from R¹⁰. In some cases, R¹ can be C₆₋₁₀ aryl, optionally substituted with 1 or 2 independently selected from R¹⁰; and R² can be C₁₋₆ haloalkyl. In some cases, R¹⁰ can be selected from halo, CN, C(O)NR^{c1}R^{d1}, C(O)OR^{a1} NR^{c1}R^{d1}, NR^{c1}C(O)R^{b1}, NR^{c1}S(O)₂R^{b1} S(O)₂R^{b1} and S(O)₂NR^{c1}R^{d1} In some cases, R¹⁰ can be selected from halo, CN, NR^{c1}R^{d1}, NR^{c1}C(O)R^{b1}, and S(O)₂R^{b1}. In some cases, R³, R⁵, and R⁶ can be each independently selected from H, halo, C₁₋₆ alkyl, C₁₋₆ haloalkyl, OR^{a1}, C(O)OR^{a1}, and NR^{c1}R^{d1}, wherein the C₁₋₆ alkyl is optionally substituted with a substituent selected from OR^{a1}, C(O)R^{b1}, C(O)NR^{c1}R^{d1}, C(O)OR^{a1}, NR^{c1}R^{d1}, S(O)₂R^{b1}, and S(O)₂NR^{c1}R^{d1}; R⁴ can be selected from Cy¹, C(O)NR^{c1}R^{d1}, C(O)OR^{a1}, NR^{c1}R^{d1}, NR^{c1}C(O)NR^{c1}R^{d1}, NR^{c1}C(S)NR^{c1}R^{d1}, and S(O)₂NR^{c1}R^{d1}; R^{c1} and R^{d1} can be each independently selected from H, C₁₋₆ alkyl, C₁₋₄ haloalkyl, C₂₋₆ alkenyl, and C₂₋₆ alkynyl, wherein the C₁₋₆ alkyl, C₂₋₆ alkenyl, and C₂₋₆ alkynyl are each optionally substituted with a substituent selected from OH, thio, C₁₋₆ alkoxy, C₁₋₆ thialkoxy, C₁₋₆ haloalkoxy, amino, C₁₋₆ alkylamino, and di(C₁₋₆ alkyl)amino (or R^{c1} and R^{d1} together with the N atom to which they are attached can form a 4-7 membered heterocycloalkyl, which is optionally substituted with 1 or 2 substituents independently selected from R^{g}); R^{a1} can be selected from H, C₁₋₆ alkyl, C₁₋₄ haloalkyl, C₂₋₆ alkenyl, and C₂₋₆ alkynyl, wherein the C₁₋₆ alkyl, C₂₋₆ alkenyl, and C₂₋₆ alkynyl are each optionally substituted with a substituent selected from OH, thio, C₁₋₆ alkoxy, C₁₋₆ thialkoxy, C₁₋₆ haloalkoxy, amino, C₁₋₆ alkylamino, and di(C₁₋₆ alkyl)amino; Cy¹ can be 5-10 membered heteroaryl, which is optionally substituted with 1 or 2 independently selected R^{Cy1}; R^{Cy1} can be selected from C₁₋₆ alkyl, C₁₋₆ haloalkyl, C₂₋₆ alkenyl, and C₂₋₆ alkynyl, each of which is optionally substituted with 1 or 2 independently selected R⁸; R⁸ can be selected from OR^{a1}, C(O)R^{b1}, C(O)NR^{c1}R^{d1}, C(O)OR^{a1} NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; R¹ and R² can be each independently selected from C₁₋₆ haloalkyl, C₆₋₁₀ aryl, and 5-10 membered heteroaryl, wherein the C₆₋₁₀ aryl and 5-10 membered heteroaryl are each optionally substituted with 1 or 2 independently selected from R¹⁰; and R¹⁰ can be selected from halo, CN, C(O)NR^{c1}R^{d1}, C(O)OR^{a1}, NR^{c1}R^{d1}, NR^{c1}C(O)R^{b1}, NR^{c1}S(O)₂R^{b1}, S(O)₂R^{b1}, and S(O)₂NR^{c1}R^{d1}. In some cases, R³, R⁵, and R⁶ can be each independently selected from H, halo, C₁₋₆ alkyl, C₁₋₆ haloalkyl, C₁₋₆ alkoxy, and C₁₋₆ haloalkoxy; and R⁴ can be selected from Cy¹, C(O)NR^{c1}R^{d1}, and C(O)OR^{a1}; R^{c1} and R^{d1} can be each independently selected from H, C₁₋₆ alkyl, and C₂₋₆ alkynyl, wherein the C₁₋₆ alkyl is optionally substituted with a substituent selected from OH, C₁₋₆ alkoxy, and di(C₁₋₆ alkyl)amino (or R^{c1} and R^{d1} together with the N atom to which they are attached can form piperazinyl, optionally substituted with 1 or 2 substituents independently selected from R^{g}); R^{a1} can be selected from H and C₁₋₆ alkyl; Cy¹ can be selected from 1,2,4-triazolyl, tetrazolyl, oxazolyl, isoxazolyl, thiophenyl, indolyl, pyrimidinyl, pyrrolopyridinyl, benzoxadiazolyl, 1,3,4-oxadiazolyl, 1,2,3-triazolyl, 1,2,4-oxadiazolyl, thiazolyl, pyridinyl, benzoxazinyl, pyrazolyl, and indazolyl, each of which is optionally substituted with 1 or 2 substituents independently selected from R^{Cy1}; R^{Cy1} can be C₁₋₆ alkyl, optionally substituted with R⁸; R⁸ can be selected from OH, thio, C₁₋₆ alkoxy, C₁₋₆ thialkoxy, C₁₋₆ haloalkoxy, amino, C₁₋₆ alkylamino, and di(C₁₋₆ alkyl)amino; R¹ and R² can be each independently selected from C₁₋₆ haloalkyl, C₆₋₁₀ aryl, and 5-10 membered heteroaryl, wherein the C₆₋₁₀ aryl and 5-10 membered heteroaryl are each optionally substituted with 1 or 2 independently selected from R¹⁰; and R¹⁰ can be selected from halo, CN, NR^{c1}R^{d1}, NR^{c1}C(O)R^{b1}, and S(O)₂R^{b1}.

In some cases, the compound of Formula (I) can be selected from any one of the compounds listed in Table 1, Table 2, Table 3a, Table 3b, Table 4, Table 5, Table 6, or Table 7, or a pharmaceutically acceptable salt thereof.

In another aspect, this document features a method of treating a mammal having a disease, disorder, or condition responsive to increasing NAMPT activity within the mammal. The method comprises (or consists essentially of or consists of) administering, to the mammal, a therapeutically effective amount of a compound of Formula (I) as recited above, or a pharmaceutically acceptable salt thereof. The mammal can be a mammal having a traumatic nerve injury, a neuropathy, a neurodegenerative condition, a central demyelinating disorder, a peripheral demyelinating disorder, a primarily inflammatory neuropathy, glaucoma, an ischemic injury, a retinal or optic nerve ischemia, a stroke, age-related vision or hearing loss, hepatosteatosis, an insulin resistance syndrome, obesity, sarcopenia, a disorder of inflammation or auto-immunity, skin aging, a cardiovascular disease, heart failure, an acute or chronic kidney injury, or infertility.

In another aspect, this document features a compound selected from BC20287 and any one of the compounds listed in Table 3b, Table 5, Table 6, or Table 7, or a pharmaceutically acceptable salt thereof.

In another aspect, this document features a pharmaceutical composition comprising (or consisting essentially of or consisting of) a compound selected from BC20287 and any one of the compounds listed in Table 3b, Table 5, Table 6, or Table 7, or a pharmaceutically acceptable salt thereof, and a pharmaceutically acceptable carrier.

In another aspect, this document features a method for increasing NAMPT activity within a mammal. The method comprises (or consists essentially of or consists of) administering, to the mammal, an effective amount of a compound of claim 48, or a pharmaceutically acceptable salt thereof.

In another aspect, this document features a method of treating a mammal having a disease, disorder, or condition responsive to increasing NAMPT activity within the mammal. The method comprises (or consists essentially of or consists of) administering, to the mammal, a therapeutically effective amount of a compound selected from BC20287 and any one of the compounds listed in Table 3b, Table 5, Table 6, or Table 7, or a pharmaceutically acceptable salt thereof. The mammal can be a mammal having a traumatic nerve injury, a neuropathy, a neurodegenerative condition, a central demyelinating disorder, a peripheral demyelinating disorder, a primarily inflammatory neuropathy, glaucoma, an ischemic injury, a retinal or optic nerve ischemia, a stroke, age-related vision or hearing loss, hepatosteatosis, an insulin resistance syndrome, obesity, sarcopenia, a disorder of inflammation or auto-immunity, skin aging, a cardiovascular disease, heart failure, an acute or chronic kidney injury, or infertility.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DETAILED DESCRIPTION

This document provides methods and materials for increasing NAMPT activity. For example, the document provides compounds (e.g., organic compounds) having the ability to increase NAMPT activity within cells and/or within a mammal, formulations containing one or more compounds having the ability to increase NAMPT activity within cells and/or within a mammal, methods for making one or more compounds having the ability to increase NAMPT activity within cells and/or within a mammal, methods for making formulations containing one or more compounds having the ability to increase NAMPT activity within cells and/or within a mammal, methods for increasing NAMPT activity within cells and/or within a mammal, and methods for treating mammals (e.g., humans) having a condition responsive to an increase of NAMPT activity. Suitable examples of conditions responsive to an increase of NAMPT activity within cells and/or within a mammal include, without limitation, traumatic nerve injuries (e.g., a neuronal crush injury, a traumatic brain injury, chronic traumatic encephalopathy (CTE), or concussion), neuropathies (e.g., a chemotherapeutic-induced sensory neuropathy or diabetic neuropathy), neurodegenerative diseases, disorders, or conditions (e.g., amyotrophic lateral sclerosis, multiple sclerosis, Huntington's disease, Alzheimer's disease, Parkinson's disease, Friedreich's ataxia, Lewy body disease, spinal muscular atrophy, frontotemporal dementia, or cerebellar degeneration), central demyelinating disorders (e.g., multiple sclerosis, adrenoleukodystrophy, adrenomyeloneuropathy, Leber hereditary optic neuropathy, neuromyelitis optica, or acute disseminated encephalomyelitis), peripheral demyelinating disorders (e.g., Charcot-Marie-Tooth disease or Guillain-Barre syndrome), other primarily inflammatory neuropathies (e.g., a multifocal motor neuropathy, an anti-MAG neuropathy, or a chronic inflammatory demyelinating polyneuropathy), glaucoma, ischemic injuries, retinal and optic nerve ischemia, and stroke. Other conditions that can be treated using one or more of the compounds described herein include, without limitation, age-related vision or hearing loss, hepatosteatosis, insulin resistance syndromes and their associated manifestations, obesity, sarcopenia, disorders of inflammation and/or auto-immunity, skin aging, skin cancer development or progression, cardiovascular diseases, heart failure, acute and/or chronic kidney injury, and infertility. In some cases, overall lifespan can be prolonged using one or more of the compounds described herein.

### Methods of Treatment Using One or More Activators of NAMPT Activity

In some cases, this document provides methods for increasing NAMPT activity within a cell by contacting the cell with one or more compounds provided herein (e.g., a compound set forth in Formula (I), or a pharmaceutically acceptable salt thereof).

In some cases, methods for increasing NAMPT activity within cells can be performed in vivo. For example, one or more compounds provided herein (e.g., a compound set forth in Formula (I), or a pharmaceutically acceptable salt thereof) can be administered to a mammal (e.g., a human) to increase NAMPT activity within cells within that mammal. In some cases, methods for increasing NAMPT activity within cells can be performed in vitro. For example, one or more compounds provided herein (e.g., a compound set forth in Formula (I), or a pharmaceutically acceptable salt thereof) can be added to a cell culture containing cells (e.g., human cells) to increase NAMPT activity within those cells. In some cases, such intervention can improve the quality of the cell while in culture or subsequently.

In some cases, this document provides methods for increasing NAMPT activity within a mammal by administering one or more compounds provided herein (e.g., a compound set forth in Formula (I), or a pharmaceutically acceptable salt thereof) to the mammal. For example, one or more cytokine activities of NAMPT can be increased within a mammal by administering one or more compounds provided herein (e.g., a compound set forth in Formula (I), or a pharmaceutically acceptable salt thereof) to the mammal.

This document also provides methods for increasing NAD⁺ levels within a cell (e.g., a neuron). Such methods can comprise (or consist essentially of or consist of) administering, to a mammal (e.g., a human) containing the cell, a therapeutically effective amount of any one or more of the compounds described herein (or one or more pharmaceutically acceptable salts thereof), or a pharmaceutical composition containing same.

This document also provides methods for treating (or preventing) a disease, disorder, or condition responsive to an increase in a NAMPT activity (thereby increasing NAD⁺ levels within a cell (e.g., a neuron)). Such methods can comprise (or consist essentially of or consist of) administering, to a mammal (e.g., a human) having the disease, disorder, or condition, a therapeutically effective amount of any one or more of the compounds described herein (or one or more pharmaceutically acceptable salts thereof), or a pharmaceutical composition comprising same.

This document also provides methods for treating diseases, disorders, and conditions in a mammal by administering one or more compounds provided herein (e.g., a compound set forth in Formula (I), or a pharmaceutically acceptable salt thereof) to a mammal in need thereof. In some cases, the disease, disorder, or condition being treated can be a disease, disorder, or condition that is responsive to increasing NAMPT activity within cells and/or within the mammal. Examples of diseases, disorders, and conditions that can be treated with one or more compounds provided herein include, without limitation, traumatic nerve injuries (e.g., a neuronal crush injury, a traumatic brain injury, chronic traumatic encephalopathy (CTE), or concussion), neuropathies (e.g., a chemotherapeutic-induced sensory neuropathy or diabetic neuropathy), neurodegenerative diseases, disorders, or conditions (e.g., amyotrophic lateral sclerosis, multiple sclerosis, Huntington's disease, Alzheimer's disease, Parkinson's disease, Friedreich's ataxia, Lewy body disease, spinal muscular atrophy, frontotemporal dementia, or cerebellar degeneration), central demyelinating disorders (e.g., multiple sclerosis, adrenoleukodystrophy, adrenomyeloneuropathy, Leber hereditary optic neuropathy, neuromyelitis optica, or acute disseminated encephalomyelitis), peripheral demyelinating disorders (e.g., Charcot-Marie-Tooth disease or Guillain-Barre syndrome), other primarily inflammatory neuropathies (e.g., a multifocal motor neuropathy, an anti-MAG neuropathy, or a chronic inflammatory demyelinating polyneuropathy), glaucoma, ischemic injuries, retinal and optic nerve ischemia, and stroke. Additional examples of diseases, disorders, and conditions that can be treated with one or more compounds provided herein include, without limitation, conditions pre-disposing to age-related vision or hearing loss, hepatosteatosis (e.g., NAFLD or NASH), insulin resistance syndromes and their associated manifestations (e.g., diabetic neuropathy, diabetic nephropathy, and diabetic retinopathy), obesity, sarcopenia and other acquired or genetic diseases of muscle weakness (e.g., inherited muscular dystrophies), disorders of inflammation and/or auto-immunity (e.g., systemic lupus erythematosus, rheumatoid arthritis, and related conditions), skin aging, skin cancer (e.g., basal cell carcinoma) development or progression, cardiovascular diseases (e.g., heart failure, atherosclerotic vascular disease, or related complications), acute or chronic kidney injuries (e.g., acute or chronic kidney injury initiated by genetic pre-disposition or environmental exposure), and infertility (e.g., female infertility or male infertility). In some cases, overall lifespan can be prolonged using one or more of the compounds described herein. In some cases, skin cancer (e.g., basal cell carcinoma) can be prevented using one or more of the compounds described herein.

In some cases, one or more compounds provided herein (e.g., a compound set forth in Formula (I), or a pharmaceutically acceptable salt thereof) can be used as described herein (e.g., to increase NAMPT activity within cells and/or within a mammal and/or to treat a disease, disorder, or condition as described herein) as the sole active ingredient(s). For example, a composition containing a compound set forth in Formula (I), or a pharmaceutically acceptable salt thereof, can lack any other active ingredients that increase NAMPT activity within cells and/or a mammal. In some cases, a composition containing a compound set forth in Formula (I), or a pharmaceutically acceptable salt thereof, can lack any other active ingredients that are effective to treat a disease, disorder, or condition as described herein.

### Therapeutic Compounds

As described herein, any one or more of the compounds provided herein (a) can be used to increase NAMPT activity within cells and/or within a mammal and/or (b) can be used to treat (or prevent) a disease, disorder, and condition in a mammal (e.g., a human) as described herein.

Formula (I)

In some embodiments, the present disclosure provides a compound of Formula (I):

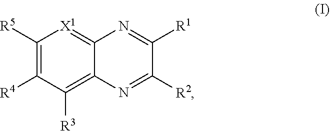

or a pharmaceutically acceptable salt thereof, wherein X¹, R¹, R², R³, R⁴, and R⁵ are as described herein.

In some embodiments:
X¹ is selected from N and CR⁶;
R³, R⁴, R⁵, and R⁶ are each independently selected from H, Cy¹, halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, C(NR^{e1})NR^{c1}R^{d1}, C(NR^{e1})NR^{c1}OR^{d1}, NR^{c1}C(O)NR^{c1}R^{d1}, NR^{c1}C(S)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from R⁷;
each R⁷ is independently selected from Cy¹, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
each Cy¹ is independently selected from C₆₋₁₀ aryl, C₃₋₁₀ cycloalkyl, 5-10 membered heteroaryl, and 4-10 membered heterocycloalkyl, each of which is optionally substituted with 1, 2, 3, 4, or 5 substituents independently selected from R^{Cy1};
each R^{Cy1} is independently selected from halo, CN, NO₂, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 5-10 membered heteroaryl, 4-10 membered heterocycloalkyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1} wherein said C₁₋₆ alkyl, C₂₋₆ alkenyl, C₂₋₆ alkynyl, C₃₋₁₀ cycloalkyl, C₆₋₁₀ aryl, 5-10 membered heteroaryl, and 4-10 membered heterocycloalkyl are each optionally substituted with 1, 2, or 3 substituents independently selected from R⁸;
each R⁸ is independently selected from C₁₋₆ alkyl, C₂₋₆ alkenyl, C₂₋₆ alkynyl, C₃₋₁₀ cycloalkyl, C₆₋₁₀ aryl, 5-10 membered heteroaryl, 4-10 membered heterocycloalkyl, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}, wherein said C₁₋₆ alkyl, C₂₋₆ alkenyl, C₂₋₆ alkynyl, C₃₋₁₀ cycloalkyl, C₆₋₁₀ aryl, 5-10 membered heteroaryl, and 4-10 membered heterocycloalkyl are each optionally substituted with 1, 2, 3, 4, or 5 substituents independently selected from R^{g};
R¹ and R² are each independently selected from R⁹ and S(O)₂R⁹;
each R⁹ is independently selected from C₁₋₆ haloalkyl, C₆₋₁₀ aryl, 5-10 membered heteroaryl, and 4-10 membered heterocycloalkyl, each of which is optionally substituted with 1, 2, 3, 4, or 5 substituents independently selected from R¹⁰;
each R¹⁰ is independently selected from halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from R¹¹;
each R¹¹ is independently selected from CN, NO₂, OR^{a1}, C(O)R^{b1}, C(O)NR^{c1}R^{d1}, C(O)OR^{a1} NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
each R^{e1} is selected from H, OR^{a1} NR^{c1}R^{d1} and C₁₋₄ haloalkyl;
each R^{a1}, R^{b1}, R^{c1}, and R^{d1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, 3, 4, or 5 substituents independently selected from R^{g};
or any R^{c1} and R^{d1} together with the N atom to which they are attached form a 4-7 membered heterocycloalkyl, which is optionally substituted with 1, 2, or 3 substituents independently selected from R^{g}; and
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkenylcarbonyl, C₁₋₆ alkynylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, and any C₁₋₆ alkyl, C₁₋₆ alkoxy, C₆₋₁₀ aryl, C₃₋₁₀ cycloalkyl, 5-10 membered heteroaryl, or 4-10 membered heterocycloalkyl of R^{g} is optionally substituted with 1, 2, or 3 substituents independently selected from OH, NO₂, CN, halo, C₁₋₆ alkyl, C₁₋₄ haloalkyl, C₁₋₆ alkoxy, and C₁₋₆ haloalkoxy.

In some embodiments, X¹ is N.

In some embodiments, X¹ is CR⁶.

In some embodiments, X¹ is CH.

In some embodiments, R³, R⁴, R⁵, and R⁶ are each independently selected from H, Cy¹, halo, CN, NO₂, C₁₋₆ alkyl, C₁₋₆ haloalkyl, OR^{a1}, C(O)NR^{c1}R^{d1}, C(O)OR^{a1}, NR^{c1}R^{d1}, NR^{c1}C(O)NR^{c1}R^{d1}, NR^{c1}C(S)NR^{c1}R^{d1}, and S(O)₂NR^{c1}R^{d1}, wherein said C₁₋₆ alkyl is optionally substituted with 1 or 2 substituents independently selected from R⁷.

In some embodiments, R³ is selected from H, Cy¹, halo, C₁₋₆ alkyl, C₁₋₆ haloalkyl, OR^{a1}, C(O)OR^{a1}, and NR^{c1}R^{d1}, wherein said C₁₋₆ alkyl is optionally substituted with a substituent selected from OR^{a1}, C(O)R^{b1}, C(O)NR^{c1}R^{d1}, C(O)OR^{a1}, NR^{c1}R^{d1}, S(O)₂R^{b1}, and S(O)₂NR^{c1}R^{d1}.

In some embodiments, R⁴ is selected from H, Cy¹, halo, C₁₋₆ alkyl, C₁₋₆ haloalkyl, OR^{a1}, C(O)OR^{a1}, and NR^{c1}R^{d1}, wherein said C₁₋₆ alkyl is optionally substituted with a substituent selected from OR^{a1}, C(O)R^{b1}, C(O)NR^{c1}R^{d1}, C(O)OR^{a1}, NR^{c1}R^{d1}, S(O)₂R^{b1}, and S(O)₂NR^{c1}R^{d1}.

In some embodiments, R⁵ is selected from H, Cy¹, halo, C₁₋₆ alkyl, C₁₋₆ haloalkyl, OR^{a1}, C(O)OR^{a1}, and NR^{c1}R^{d1}, wherein said C₁₋₆ alkyl is optionally substituted with a substituent selected from OR^{a1}, C(O)R^{b1}, C(O)NR^{c1}R^{d1}, C(O)OR^{a1}, NR^{c1}R^{d1}, S(O)₂R^{b1}, and S(O)₂NR^{c1}R^{d1}.

In some embodiments, R⁶ is selected from H, Cy¹, halo, C₁₋₆ alkyl, C₁₋₆ haloalkyl, OR^{a1}, C(O)OR^{a1}, and NR^{c1}R^{d1}, wherein said C₁₋₆ alkyl is optionally substituted with a substituent selected from OR^{a1}, C(O)R^{b1}, C(O)NR^{c1}R^{d1}, C(O)OR^{a1}, NR^{c1}R^{d1}, S(O)₂R^{b1}, and S(O)₂NR^{c1}R^{d1}.

In some embodiments, R³, R⁵, and R⁶ are each independently selected from H, halo, C₁₋₆ alkyl, C₁₋₆ haloalkyl, OR^{a1}, C(O)OR^{a1}, and NR^{c1}R^{d1}, wherein said C₁₋₆ alkyl is optionally substituted with a substituent selected from OR^{a1}, C(O)R^{b1}, (O)NR^{c1}R^{d1}, C(O)OR^{a1}, NR^{c1}R^{d1}, S(O)₂R^{b1}, and S(O)₂NR^{c1}R^{d1}.

In some embodiments, R³, R⁵, and R⁶ are each independently selected from H, halo, C₁₋₆ alkyl, C₁₋₆ haloalkyl, C₁₋₆ alkoxy, and C₁₋₆ haloalkoxy.

In some embodiments, R³, R⁵, and R⁶ are each selected from H and C₁₋₆ alkyl.

In some embodiments, R³, R⁵, and R⁶ are each H.

In some embodiments, R⁴ is selected from Cy¹, C(O)NR^{c1}R^{d1}, C(O)OR^{a1}. NR^{c1}R^{d1}, NR^{c1}C(O)NR^{c1}R^{d1}, NR^{c1}C(S)NR^{c1}R^{d1}, and S(O)₂NR^{c1}R^{d1}.

In some embodiments, R⁴ is selected from Cy¹, C(O)NR^{c1}R^{d1}, and C(O)OR^{a1}.

In some embodiments, R⁴ is Cy¹ (e.g., 1,2,4-triazolyl, tetrazolyl, oxazolyl, or 1,2,4-oxadiazolyl, each of which is optionally substituted with 1 or 2 substituents independently selected from R^{Cy1}).

In some embodiments, R⁴ is C(O)NR^{c1}R^{d1}.

In some embodiments, R⁴ is a group of formula:

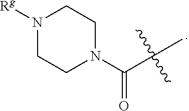

In some embodiments, R⁴ is C(O)OR^{a1} (e.g., R¹ is H or C₁₋₆ alkyl).

In some embodiments, the compound of Formula (I) has formula:

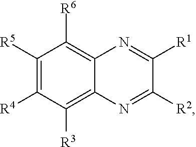

or a pharmaceutically acceptable salt thereof.

In some embodiments:
R³, R⁵, and R⁶ are each independently selected from H, halo, C₁₋₆ alkyl, C₁₋₆ haloalkyl, OR^{a1}, C(O)OR^{a1}, and NR^{c1}R^{d1}, wherein said C₁₋₆ alkyl is optionally substituted with a substituent selected from OR^{a1}, C(O)R^{b1}, C(O)NR^{c1}R^{d1}, C(O)OR^{a1}, NR^{c1}R^{d1}, S(O)₂R^{b1}, and S(O)₂NR^{c1}R^{d1}; and
R⁴ is selected from Cy¹, C(O)NR^{c1}R^{d1}, C(O)OR^{a1}, NR^{c1}R^{d1}, NR^{c1}C(O)NR^{c1}R^{d1}, NR^{c1}C(S)NR^{c1}R^{d1}, and S(O)₂NR^{c1}R^{d1}.

In some embodiments:
R³, R⁵, and R⁶ are each independently selected from H, halo, C₁₋₆ alkyl, C₁₋₆ haloalkyl, C₁₋₆ alkoxy, C₁₋₆ haloalkoxy; and
R⁴ is selected from Cy¹, C(O)NR^{c1}R^{d1}, and C(O)OR^{a1}.

In some embodiments, the compound of Formula (I) has formula:

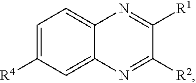

or a pharmaceutically acceptable salt thereof.

In some embodiments, the compound of Formula (I) has formula:

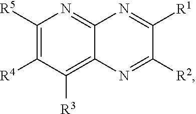

or a pharmaceutically acceptable salt thereof.

In some embodiments:
R³ and R⁵ are each independently selected from H, halo, C₁₋₆ alkyl, C₁₋₆ haloalkyl, OR^{a1}, C(O)OR^{a1}, and NR^{c1}R^{d1}, wherein said C₁₋₆ alkyl is optionally substituted with a substituent selected from OR^{a1}, C(O)R^{b1}, C(O)NR^{c1}R^{d1}, C(O)OR^{a1}, NR^{c1}R^{d1}, S(O)₂R^{b1} and S(O)₂NR^{c1}R^{d1}; and
R⁴ is selected from Cy¹, C(O)NR^{c1}R^{d1}, C(O)OR^{a1}, NR^{c1}R^{d1}, NR^{c1}C(O)NR^{c1}R^{d1}, NR^{c1}C(S)NR^{c1}R^{d1}, and S(O)₂NR^{c1}R^{d1}.

In some embodiments:
R³ and R⁵ are each independently selected from H, halo, C₁₋₆ alkyl, C₁₋₆ haloalkyl, C₁₋₆ alkoxy, C₁₋₆ haloalkoxy; and
R⁴ is selected from Cy¹, C(O)NR^{c1}R^{d1}, and C(O)OR^{a1}.

In some embodiments, the compound of Formula (I) has formula:

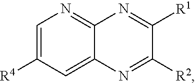

or a pharmaceutically acceptable salt thereof.

In some embodiments, the compound of Formula (I) has formula:

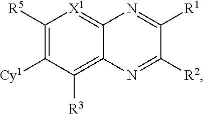

or a pharmaceutically acceptable salt thereof.

In some embodiments, the compound of Formula (I) has formula:

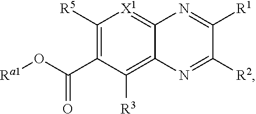

or a pharmaceutically acceptable salt thereof.

In some embodiments, the compound of Formula (I) has formula:

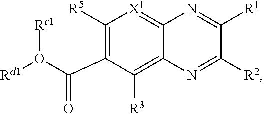

or a pharmaceutically acceptable salt thereof.

In some embodiments, the compound of Formula (I) has formula:

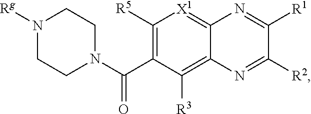

or a pharmaceutically acceptable salt thereof.

In some embodiments, R^{c1} and R^{d1} are each independently selected from H, C₁₋₆ alkyl, C₁₋₄ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with a substituent selected from OH, thio, alkoxy, C₁₋₆ thialkoxy, C₁₋₆ haloalkoxy, amino, C₁₋₆ alkylamino, and di(C₁₋₆ alkyl)amino.

In some embodiments, R^{c1} and R^{d1} are each independently selected from H, C₁₋₆ alkyl, and C₂₋₆ alkynyl, wherein said C₁₋₆ alkyl is optionally substituted with a substituent selected from OH, C₁₋₆ alkoxy, and di(C₁₋₆ alkyl)amino.

In some embodiments, R^{c1} and R^{d1} together with the N atom to which they are attached form a 4-7 membered heterocycloalkyl, which is optionally substituted with 1 or 2 substituents independently selected from R^{g}.

In some embodiments, R^{c1} is H and R^{d1} is C₂₋₆ alkynyl.

In some embodiments, R^{c1} and R^{d1} together with the N atom to which they are attached form a ring selected from morpholinyl, thiomorpholinyl, piperazinyl, piperidinyl, pyrrolidinyl, and azepanyl, each of which is optionally substituted with 1 or 2 substituents independently selected from R^{g}.

In some embodiments, R^{c1} and R^{d1} together with the N atom to which they are attached form a piperazinyl ring, optionally substituted with 1 or 2 R^{g}.

In some embodiments, R^{c1} and R^{d1} together with the N atom to which they are attached form a piperazinyl ring of formula:

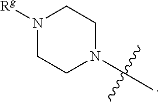

In some embodiments, R^{a1} is selected from H, C₁₋₆ alkyl, C₁₋₄ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with a substituent selected from OH, thio, C₁₋₆ alkoxy, C₁₋₆ thialkoxy, C₁₋₆ haloalkoxy, amino, C₁₋₆ alkylamino, and di(C₁₋₆ alkyl)amino.

In some embodiments, R^{a1} is selected from H, C₁₋₆ alkyl, and C₂₋₆ alkynyl, wherein said C₁₋₆ alkyl is optionally substituted with a substituent selected from OH, C₁₋₆ alkoxy, and di(C₁₋₆ alkyl)amino.

In some embodiments, R^{a1} is selected from H and C₁₋₆ alkyl.

In some embodiments, R^{a1} is C₂₋₆ alkynyl.

In some embodiments:
R^{c1} and R^{d1} are each independently selected from H, C₁₋₆ alkyl, C₁₋₄ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with a substituent selected from OH, thio, C₁₋₆ alkoxy, C₁₋₆ thialkoxy, C₁₋₆ haloalkoxy, amino, C₁₋₆ alkylamino, and di(C₁₋₆ alkyl)amino; and
R^{a1} is selected from H, C₁₋₆ alkyl, C₁₋₄ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with a substituent selected from OH, thio, C₁₋₆ alkoxy, C₁₋₆ thialkoxy, C₁₋₆ haloalkoxy, amino, C₁₋₆ alkylamino, and di(C₁₋₆ alkyl)amino.

In some embodiments:
R^{c1} and R^{d1} are each independently selected from H, C₁₋₆ alkyl, and C₂₋₆ alkynyl, wherein said C₁₋₆ alkyl is optionally substituted with a substituent selected from OH, C₁₋₆ alkoxy, and di(C₁₋₆ alkyl)amino; and
R^{a1} is selected from H, C₁₋₆ alkyl, and C₂₋₆ alkynyl, wherein said C₁₋₆ alkyl is optionally substituted with a substituent selected from OH, C₁₋₆ alkoxy, and di(C₁₋₆ alkyl)amino.

In some embodiments, R^{b1} is selected from C₁₋₆ alkyl, C₁₋₄ haloalkyl, C₂₋₆ alkenyl, and C₂₋₆ alkynyl, each of which is optionally substituted with 1 or 2 independently selected R^{g}.

In some embodiments, R^{b1} is C₁₋₆ alkyl, optionally substituted with R^{g}.

In some embodiments, Cy¹ is C₆₋₁₀ aryl, optionally substituted with 1 or 2 substituents independently selected from R^{Cy1}.

In some embodiments, Cy¹ is C₃₋₁₀ cycloalkyl, optionally substituted with 1 or 2 substituents independently selected from R^{Cy1}.

In some embodiments, Cy¹ is 4-10 membered heterocycloalkyl, optionally substituted with 1 or 2 substituents independently selected from R^{Cy1}.

In some embodiments, Cy¹ is 5-10 membered heteroaryl, optionally substituted with 1 or 2 substituents independently selected from R^{Cy1}.

In some embodiments, Cy¹ is 5-6 membered heteroaryl, optionally substituted with 1 or 2 substituents independently selected from R.

In some embodiments, Cy¹ is 5-membered heteroaryl, optionally substituted with 1 or 2 substituents independently selected from R.

In some embodiments, Cy¹ is selected from 1,2,4-triazolyl, tetrazolyl, oxazolyl, isoxazolyl, thiophenyl, indolyl, pyrimidinyl, pyrrolopyridinyl, benzoxadiazolyl, 1,3,4-oxadiazolyl, 1,2,3-triazolyl, 1,2,4-oxadiazolyl, thiazolyl, pyridinyl, benzoxazinyl, pyrazolyl, and indazolyl, each of which is optionally substituted with 1 or 2 substituents independently selected from R^{Cy1}.

In some embodiments, Cy¹ is selected from 1,2,4-triazolyl, tetrazolyl, oxazolyl, and 1,2,4-oxadiazolyl, each of which is optionally substituted with 1 or 2 substituents independently selected from R^{Cy1}.

In some embodiments, R^{Cy1} is selected from C₁₋₆ alkyl, C₁₋₆ haloalkyl, C₂₋₆ alkenyl, and C₂₋₆ alkynyl, each of which is optionally substituted with 1 or 2 independently selected R⁸.

In some embodiments, R^{Cy1} is C₁₋₆ alkyl, optionally substituted with R⁸.

In some embodiments, R⁸ is selected from OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}.

In some embodiments, R⁸ is selected from OH, thio, C₁₋₆ alkoxy, C₁₋₆ thialkoxy, C₁₋₆ haloalkoxy, amino, C₁₋₆ alkylamino, and di(C₁₋₆ alkyl)amino.

In some embodiments, R¹ is R⁹.

In some embodiments, R¹ is S(O)₂R⁹.

In some embodiments, R² is R⁹.

In some embodiments, R² is S(O)₂R⁹.

In some embodiments, R¹ is R⁹ and R² is R⁹.

In some embodiments, R¹ is R⁹ and R² is S(O)₂R⁹.

In some embodiments, R¹ is S(O)₂R⁹ and R² is R⁹.

In some embodiments, R⁹ is C₁₋₆ haloalkyl.

In some embodiments, R⁹ is selected from C₆₋₁₀ aryl, 5-10 membered heteroaryl, and 4-10 membered heterocycloalkyl, each of which is optionally substituted with 1, 2, or 3 substituents independently selected from R¹⁰.

In some embodiments, R⁹ is C₆₋₁₀ aryl, optionally substituted with 1, 2, or 3 substituents independently selected from R¹⁰.

In some embodiments, R⁹ is 5-10 membered heteroaryl, optionally substituted with 1, 2, or 3 substituents independently selected from R¹⁰.

In some embodiments, R⁹ is 4-10 membered heterocycloalkyl, optionally substituted with 1, 2, or 3 substituents independently selected from R¹⁰.

In some embodiments, R¹ and R² are each independently selected from C₁₋₆ haloalkyl, C₆₋₁₀ aryl, and 5-10 membered heteroaryl, wherein said C₆₋₁₀ aryl and 5-10 membered heteroaryl are each optionally substituted with 1 or 2 independently selected from R¹⁰.

In some embodiments, R¹ and R² are each independently a C₆₋₁₀ aryl, optionally substituted with 1 or 2 independently selected from R¹⁰.

In some embodiments:
R¹ is 5-10 membered heteroaryl, optionally substituted with 1 or 2 independently selected from R¹⁰; and
R² is C₆₋₁₀ aryl, optionally substituted with 1 or 2 independently selected from R¹⁰.

In some embodiments:
R¹ is C₆₋₁₀ aryl, optionally substituted with 1 or 2 independently selected from R¹⁰; and
R² is 5-10 membered heteroaryl, optionally substituted with 1 or 2 independently selected from R¹⁰.

In some embodiments:
R¹ is C₁₋₆ haloalkyl; and
R² is C₆₋₁₀ aryl, optionally substituted with 1 or 2 independently selected from R¹⁰.

In some embodiments:
R¹ is C₁₋₆ haloalkyl; and
R² is 5-10 membered heteroaryl, optionally substituted with 1 or 2 independently selected from R¹⁰.

In some embodiments:
R¹ is C₆₋₁₀ aryl, optionally substituted with 1 or 2 independently selected from R¹⁰; and
R² is C₁₋₆ haloalkyl.

In some embodiments, R¹⁰ is selected from halo, CN, C(O)NR^{c1}R^{d1}, C(O)OR^{a1}, NR^{c1}R^{d1}, NR^{c1}C(O)R^{b1}, NR^{c1}S(O)₂R^{b1}, S(O)₂R^{b1}, and S(O)₂NR^{c1}R^{d1}.

In some embodiments, R¹⁰ is selected from halo, CN, NR^{c1}R^{d1}, NR^{c1}C(O)R^{b1}, and S(O)₂R^{b1}.

In some embodiments, R^{g} is selected from OH, halo, C₁₋₆ alkyl, C₁₋₄ haloalkyl, C₁₋₆ alkoxy, C₁₋₆ haloalkoxy, amino, C₁₋₆ alkylamino, and di(C₁₋₆ alkyl)amino.

In some embodiments:
R³, R⁵, and R⁶ are each independently selected from H, halo, C₁₋₆ alkyl, C₁₋₆ haloalkyl, OR^{a1}, C(O)OR^{a1}, and NR^{c1}R^{d1}, wherein said C₁₋₆ alkyl is optionally substituted with a substituent selected from OR^{a1}, C(O)R^{b1}, C(O)NR^{c1}R^{d1}, C(O)OR^{a1}, NR^{c1}R^{d1} S(O)₂R^{b1}, and S(O)₂NR^{c1}R^{d1};
R⁴ is selected from Cy¹, C(O)NR^{c1}R^{d1}, C(O)OR^{a1}, NR^{c1}R^{d1}, NR^{c1}C(O)NR^{c1}R^{d1}, NR^{c1}C(S)NR^{c1}R^{d1}, and S(O)₂NR^{c1}R^{d1};
R^{c1} and R^{d1} are each independently selected from H, C₁₋₆ alkyl, C₁₋₄ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with a substituent selected from OH, thio, C₁₋₆ alkoxy, C₁₋₆ thialkoxy, C₁₋₆ haloalkoxy, amino, C₁₋₆ alkylamino, and di(C₁₋₆ alkyl)amino; or
R^{c1} and R^{d1} together with the N atom to which they are attached form a 4-7 membered heterocycloalkyl, which is optionally substituted with 1 or 2 substituents independently selected from R^{g};
R^{a1} is selected from H, C₁₋₆ alkyl, C₁₋₄ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with a substituent selected from OH, thio, C₁₋₆ alkoxy, C₁₋₆ thialkoxy, C₁₋₆ haloalkoxy, amino, C₁₋₆ alkylamino, and di(C₁₋₆ alkyl)amino;
Cy¹ is 5-10 membered heteroaryl, which is optionally substituted with 1 or 2 independently selected R^{Cy1};
R^{Cy1} is selected from C₁₋₆ alkyl, C₁₋₆ haloalkyl, C₂₋₆ alkenyl, and C₂₋₆ alkynyl, each of which is optionally substituted with 1 or 2 independently selected R⁸;
R⁸ is selected from OR^{a1}, C(O)R^{b1}, C(O)NR^{c1}R^{d1}, C(O)_{0R′1} NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
R¹ and R² are each independently selected from C₁₋₆ haloalkyl, C₆₋₁₀ aryl, and 5-10 membered heteroaryl, wherein said C₆₋₁₀ aryl and 5-10 membered heteroaryl are each optionally substituted with 1 or 2 independently selected from R¹⁰; and
R¹⁰ is selected from halo, CN, C(O)NR^{c1}R^{d1}, C(O)OR^{a1}, NR^{c1}R^{d1}, NR^{c1}C(O)R^{b1}, NR^{c1}S(O)₂R^{b1}, S(O)₂R^{b1}, and S(O)₂NR^{c1}R^{d1}.

In some embodiments:
R³, R⁵, and R⁶ are each independently selected from H, halo, C₁₋₆ alkyl, C₁₋₆ haloalkyl, C₁₋₆ alkoxy, and C₁₋₆ haloalkoxy; and
R⁴ is selected from Cy¹, C(O)NR^{c1}R^{d1} and C(O)OR^{a1};
R^{c1} and R^{d1} are each independently selected from H, C₁₋₆ alkyl, and C₂₋₆ alkynyl, wherein said C₁₋₆ alkyl is optionally substituted with a substituent selected from OH, C₁₋₆ alkoxy, and di(C₁₋₆ alkyl)amino; or
R^{c1} and R^{d1} together with the N atom to which they are attached form piperazinyl, optionally substituted with 1 or 2 substituents independently selected from R^{g};
R^{a1} is selected from H and C₁₋₆ alkyl;
Cy¹ is selected from 1,2,4-triazolyl, tetrazolyl, oxazolyl, isoxazolyl, thiophenyl, indolyl, pyrimidinyl, pyrrolopyridinyl, benzoxadiazolyl, 1,3,4-oxadiazolyl, 1,2,3-triazolyl, 1,2,4-oxadiazolyl, thiazolyl, pyridinyl, benzoxazinyl, pyrazolyl, and indazolyl, each of which is optionally substituted with 1 or 2 substituents independently selected from R^{Cy1};
R^{Cy1} is C₁₋₆ alkyl, optionally substituted with R⁸;
R⁸ is selected from OH, thio, C₁₋₆ alkoxy, C₁₋₆ thialkoxy, C₁₋₆ haloalkoxy, amino, C₁₋₆ alkylamino, and di(C₁₋₆ alkyl)amino;
R¹ and R² are each independently selected from C₁₋₆ haloalkyl, C₆₋₁₀ aryl, and 5-10 membered heteroaryl, wherein said C₆₋₁₀ aryl and 5-10 membered heteroaryl are each optionally substituted with 1 or 2 independently selected from R¹⁰; and
R¹⁰ is selected from halo, CN, NR^{c1}R^{d1}, NR^{c1}C(O)R^{b1}, and S(O)₂R^{b1}.

In some embodiments, the compound of Formula (I) is selected from any one of the compounds listed in Table 1, Table 2, Table 3a, Table 3b, Table 4, Table 5, Table 6, or Table 7, or a pharmaceutically acceptable salt thereof.

In some embodiments, the compound of Formula (I) is selected from any one of the compounds listed in Table 1, or a pharmaceutically acceptable salt thereof.

In some embodiments, the compound of Formula (I) is selected from any one of the compounds listed in Table 2, or a pharmaceutically acceptable salt thereof.

In some embodiments, the compound of Formula (I) is selected from any one of the compounds listed in Table 3a, or a pharmaceutically acceptable salt thereof.

In some embodiments, the compound of Formula (I) is selected from any one of the compounds listed in Table 3b, or a pharmaceutically acceptable salt thereof.

In some embodiments, the compound of Formula (I) is selected from any one of the compounds listed in Table 4, or a pharmaceutically acceptable salt thereof.

In some embodiments, the compound of Formula (I) is selected from any one of the compounds listed in Table 5, or a pharmaceutically acceptable salt thereof.

In some embodiments, the compound of Formula (I) is selected from any one of the compounds listed in Table 6, or a pharmaceutically acceptable salt thereof.

In some embodiments, the compound of Formula (I) is selected from any one of the compounds listed in Table 7, or a pharmaceutically acceptable salt thereof.

In some embodiments, a salt of any one of the compounds disclosed herein is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds disclosed herein include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and those formed with organic acids such as maleic acid.

In some embodiments, bases commonly employed to form pharmaceutically acceptable salts of the compounds disclosed herein include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, any one of the compounds disclosed herein, or a pharmaceutically acceptable salt thereof, is substantially isolated.

### Methods of Making Therapeutic Compounds

Compounds as set forth in any one of the Formulae disclosed herein, including salts thereof, can be prepared using organic synthesis techniques and can be synthesized according to any of numerous possible synthetic routes. A person skilled in the art knows how to select and implement appropriate synthetic protocols, and appreciates that a broad repertoire of synthetic organic reactions is available to be potentially employed in synthesizing compounds provided herein.

Suitable synthetic methods of starting materials, intermediates, and products can be identified by reference to the literature, including reference sources such as: Advances in Heterocyclic Chemistry, Vols. 1-107 (Elsevier, 1963-2012); Journal of Heterocyclic Chemistry Vols. 1-49 (J. Heterocyclic Chemistry, 1964-2012); Carreira et al., (Ed.) Science of Synthesis, Vols. 1-48 (2001-2010) and Knowledge Updates KU2010/1-4; 2011/1-4; 2012/1-2 (Thieme, 2001-2012); Katritzky et al., (Ed.) Comprehensive Organic Functional Group Transformations, (Pergamon Press, 1996); Katritzky et al., (Ed.) Comprehensive Organic Functional Group Transformations II (Elsevier, 2^{nd} Edition, 2004); Katritzky et al., (Ed.) Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.) Comprehensive Organic Synthesis (Pergamon Press, 1991).

The reactions for preparing the compounds provided herein can be carried out in suitable solvents that can be readily selected by one of skill in the art of organic synthesis. Suitable solvents can be substantially non-reactive with the starting materials (reactants), the intermediates, or products at the temperatures at which the reactions are carried out, e.g., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in P. G. M. Wuts and T. W. Greene, Protective Groups in Organic Synthesis, 4^{th} Ed., Wiley & Sons, Inc., New York (2006).

### Pharmaceutical Compositions and Formulations

This document also provides pharmaceutical compositions comprising an effective amount of a compound disclosed herein, or a pharmaceutically acceptable salt thereof; and a pharmaceutically acceptable carrier. The pharmaceutical composition also can comprise any one of the additional therapeutic agents and/or therapeutic molecules described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants, and vehicles that can be used in the pharmaceutical compositions provided herein include, without limitation, ion exchangers, alumina, aluminum stearate, lecithin, serum proteins (e.g., human serum albumin), buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms can contain any one or more of the compounds or therapeutic agents described herein in the range of 0.005 percent to 100 percent with the balance made up from the suitable pharmaceutically acceptable carriers or excipients. The contemplated compositions can contain from about 0.001 percent to about 100 percent (e.g., from about 0.1 percent to about 95 percent, from about 75 percent to about 85 percent, or from about 20 percent to about 80 percent) of any one or more of the compounds or therapeutic agents provided herein, wherein the balance can be made up of any pharmaceutically acceptable carrier or excipient described herein, or any combination of these carriers or excipients.

### Routes of Administration and Dosage Forms

The therapeutic compounds and/or pharmaceutical compositions provided herein (e.g., a composition containing one or more compounds disclosed herein, or a pharmaceutically acceptable salt thereof) can include those suitable for any acceptable route of administration. Acceptable routes of administration include, without limitation, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral, vaginal, intravitreal, subretinal or other intraocular routes of administrations.

Compositions and formulations described herein can conveniently be presented in a unit dosage form, e.g., tablets, sustained release capsules, and in liposomes, and can be prepared by any methods well known in the art of pharmacy. See, for example, Remington: The Science and Practice of Pharmacy, Lippincott Williams & Wilkins, Baltimore, MD (20th ed. 2000). Such preparative methods include, without limitation, the step of bringing into association with the molecule to be administered ingredients such as a carrier that constitutes one or more accessory ingredients. In general, the compositions can be prepared by uniformly and intimately bringing into association the active ingredients with liquid carriers, liposomes, or finely divided solid carriers, or both, and then, if necessary, shaping the product.

In some embodiments, any one or more of the compounds or therapeutic agents described herein can be administered orally. Compositions described herein that are suitable for oral administration can be presented as discrete units such as capsules, sachets, granules, or tablets each containing a predetermined amount (e.g., effective amount) of the active ingredient(s); a powder or granules; a solution or a suspension in an aqueous liquid or a non-aqueous liquid; an oil-in-water liquid emulsion; a water-in-oil liquid emulsion; packed in liposomes; or as a bolus. Soft gelatin capsules can be useful for containing such suspensions, which can beneficially increase the rate of compound absorption. In the case of tablets for oral use, carriers that are commonly used include, without limitation, lactose, sucrose, glucose, mannitol, silicic acid, and starches. Other acceptable excipients can include, without limitation, (a) fillers or extenders such as starches, lactose, sucrose, glucose, mannitol, and silicic acid, (b) binders such as carboxymethylcellulose, alginates, gelatin, polyvinylpyrrolidone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. For oral administration in a capsule form, useful diluents include, without limitation, lactose and dried cornstarch. When aqueous suspensions are administered orally, the active ingredient(s) can be combined with emulsifying and suspending agents. If desired, certain sweetening and/or flavoring and/or coloring agents can be added. Compositions suitable for oral administration include, without limitation, lozenges comprising ingredients in a flavored basis, usually sucrose and acacia or tragacanth; and pastilles comprising the active ingredient(s) in an inert basis such as gelatin and glycerin, or sucrose and acacia.

Compositions suitable for parenteral administration include, without limitation, aqueous and non-aqueous sterile injection solutions or infusion solutions that may contain antioxidants, buffers, bacteriostats, and solutes that render the formulation isotonic with the blood of the intended recipient; and aqueous and non-aqueous sterile suspensions that may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water, for injections, saline (e.g., 0.9% saline solution), or 5% dextrose solution, immediately prior to use. Extemporaneous injection solutions and suspensions can be prepared from sterile powders, granules, and tablets. The injection solutions can be in the form of, for example, a sterile injectable aqueous or oleaginous suspension. This suspension can be formulated according to techniques known in the art using suitable dispersing or wetting agents and suspending agents. A sterile injectable preparation also can be a sterile injectable solution or suspension in a non-toxic parenterally-acceptable diluent or solvent, for example, as a solution in 1,3-butanediol. Among the acceptable vehicles and solvents that can be employed are mannitol, water, Ringer's solution, and isotonic sodium chloride solution. In addition, sterile, fixed oils can be used as a solvent or suspending medium. For this purpose, any bland fixed oil can be used including, without limitation, synthetic mono- or diglycerides. Fatty acids such as oleic acid and its glyceride derivatives can be used to prepare injectables. In some cases, natural pharmaceutically acceptable oils such as olive oil or castor oil, especially in their polyoxyethylated versions, can be used to prepare injectables. These oil solutions or suspensions also can contain a long-chain alcohol diluent or dispersant.

In some cases, a therapeutic compound and/or pharmaceutical composition provided herein can be administered in the form of suppository for rectal administration. These compositions can be prepared by mixing a compound described herein (e.g., any one of the compounds disclosed herein, or a pharmaceutically acceptable salt thereof) with a suitable non-irritating excipient that is solid at room temperature but liquid at the rectal temperature and therefore will melt in the rectum to release the active component(s). Such materials include, without limitation, cocoa butter, beeswax, and polyethylene glycols.

In some cases, a therapeutic compounds and/or pharmaceutical composition provided herein can be administered by nasal aerosol or inhalation. Such compositions can be prepared according to techniques well known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J. Pharm. Pharmacol., 56:3-17 (2004); and Ilium, L., Eur. J. Pharm. Sci., 11:1-18 (2000).

In some cases, a therapeutic compounds and/or pharmaceutical composition provided herein can be prepared as a topical composition and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of a therapeutic compounds and/or pharmaceutical composition provided herein can be useful when the desired treatment involves areas or organs readily accessible by topical application. In some cases, a topical composition can include a combination of any one or more of the compounds or therapeutic agents described herein (e.g., a compound set forth in any one of Formulae disclosed herein, or a pharmaceutically acceptable salt thereof), and one or more additional ingredients, carriers, excipients, or diluents including, without limitation, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

In some cases, one or more compounds or therapeutic agent described herein (e.g., any one of the compounds disclosed herein, or a pharmaceutically acceptable salt thereof) can be incorporated into a composition for coating an implantable medical device such as a prosthesis, artificial valve, vascular graft, stent, or catheter. Suitable coatings and the general preparation of coated implantable devices are known in the art and are exemplified in U.S. Pat. Nos. 6,099,562; 5,886,026; and 5,304,121. The coatings can be biocompatible polymeric materials such as a hydrogel polymer, polymethyldisiloxane, polycaprolactone, polyethylene glycol, polylactic acid, ethylene vinyl acetate, or mixture thereof. In some cases, the coating can optionally be further covered by a suitable topcoat of fluorosilicone, polysaccharides, polyethylene glycol, phospholipids or combinations thereof to impart controlled release characteristics in the composition.

In some cases, this document provides an implantable drug release device impregnated with or containing one or more compounds or therapeutic agents described herein (e.g., any one of the compounds disclosed herein, or a pharmaceutically acceptable salt thereof) such that the compound(s) or therapeutic agent(s) are released from the device and are therapeutically active.

### Dosages and Regimens

A composition (e.g., pharmaceutical compositions provided herein) containing a compound provided herein, or a pharmaceutically acceptable salt thereof, can include that compound in an effective amount (e.g., a therapeutically effective amount).

Effective doses can vary, depending on the diseases being treated, the severity of the disease, the route of administration, the sex, age and general health condition of the subject, excipient usage, the possibility of co-usage with other therapeutic treatments such as use of other agents, and the judgment of the treating physician.

In some embodiments, an effective amount of a compound as disclosed herein, or a pharmaceutically acceptable salt thereof, can range, for example, from about 0.1 mg to about 1000 mg. In some cases, the effective amount can be from about 0.5 mg to about 500 mg of a compound disclosed herein, or any amount in between these two values, for example, one of about 0.5 mg, about 1 mg, about 2 mg, about 5 mg, about 10 mg, about 20 mg, about 50 mg, about 100 mg, about 200 mg, about 250 mg, about 300 mg, about 400 mg, or about 500 mg. The effective amount can be an amount sufficient to alleviate or reduce one or more of the symptoms associated with a disease, disorder, or condition being treated as described herein.

In some cases, an effective amount of a compound as disclosed herein, or a pharmaceutically acceptable salt thereof, can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; from about 0.1 mg/kg to about 0.5 mg/kg, or from about 0.5 mg/kg to about 500 mg/kg).

In some cases, an effective amount of a compound as disclosed herein, or a pharmaceutically acceptable salt thereof, can be about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or on a non-daily basis (e.g., every other day, every two days, every three days, once weekly, twice weekly, once every two weeks, or once a month). In some cases, the dosages can be administered every 4 hours, 6 hours, 8 hours, 12 hours, or 24 hours.

### Kits

This document also provides pharmaceutical kits useful, for example, to activate NAMPT within cells within a mammal (e.g., a human). In some cases, this document provides pharmaceutical kits useful, for example, to treat diseases, disorders, and conditions referred to herein. Such pharmaceutical kits can include one or more containers containing a pharmaceutical composition that includes a therapeutically effective amount of a compound provided herein, or a pharmaceutically acceptable salt thereof. In some cases, such kits can further include, if desired, one or more of various conventional pharmaceutical kit components such as containers with one or more pharmaceutically acceptable carriers. Instructions, either as inserts or as labels, indicating quantities of the components to be administered, guidelines for administration, and/or guidelines for mixing the components also can be included in a kit provided herein.

### Combination Therapies

In some cases, one or more compounds provided herein, or a pharmaceutically acceptable salt thereof, can be combined with one or more therapeutic molecules. Examples of therapeutic molecules that can be used in combination with one or more compounds provided herein, or a pharmaceutically acceptable salt thereof, include, without limitation, nicotinamide riboside, nicotinamide mononucleotide, NAD precursor molecules, inhibitors of NAD degradation (e.g., CD38 inhibitors), and inhibitors of NAD consumption (e.g., PARP inhibitors).

One or more compounds provided herein, or a pharmaceutically acceptable salt thereof, and the one or more therapeutic molecules can be administered in any order or simultaneously. If simultaneously administered, they can be provided in a single, unified, form or in multiple forms (e.g., either as a single pill or as two separate pills). One of the items can be given in multiple doses, or both can be given as multiple doses. If not simultaneous, the timing between the multiple doses can vary from more than zero weeks to less than four weeks.

### Definitions

As used herein, the term “about” means “approximately” (e.g., plus or minus approximately 10% of the indicated value).

At various places in this document, substituents of compounds provided herein are disclosed in groups or in ranges. It is specifically intended that these groups and ranges include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in this document various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features described herein, which are, for clarity, described in the context of separate embodiments, can also be provided in combination in a single embodiment. Conversely, various features described herein which are, for brevity, described in the context of a single embodiment, also can be provided separately or in any suitable subcombination.

The term “aromatic” refers to a carbocycle or heterocycle having one or more polyunsaturated rings having aromatic character (i.e., having (4n+2) delocalized π (pi) electrons where n is an integer).

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution can be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, without limitation, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms that may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cₙ₋ₘ alkenyl” refers to an alkyl group having one or more double carbon-carbon bonds and having n to m carbons. Example alkenyl groups include, without limitation, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, without limitation, ethynyl, propyn-1-yl, propyn-2-yl, and the like. In some embodiments, the alkynyl moiety contains 2 to 6, 2 to 4, or 2 to 3 carbon atoms.

As used herein, the term “Cn-m alkylene”, employed alone or in combination with other terms, refers to a divalent alkyl-linking group having n to m carbons. Examples of alkylene groups include, without limitation, ethan-1,1-diyl, ethan-1,2-diyl, propan-1,1-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cn-m alkoxy”, employed alone or in combination with other terms, refers to a group of formula —O-alkyl, wherein the alkyl group has n to m carbons. Example alkoxy groups include, without limitation, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cn-m alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, without limitation, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “Cn-m alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms. Examples of alkoxycarbonyl groups include, without limitation, methoxycarbonyl, ethoxycarbonyl, propoxycarbonyl (e.g., n-propoxycarbonyl and isopropoxycarbonyl), butoxycarbonyl (e.g., n-butoxycarbonyl and tert-butoxycarbonyl), and the like.

As used herein, the term “Cₙ₋ₘ alkylcarbonyl” refers to a group of formula —C(O)-alkyl, wherein the alkyl group has n to m carbon atoms. In some embodiments, the alkyl group has 1 to 6, 1 to 4, or 1 to 3 carbon atoms. Examples of alkylcarbonyl groups include, without limitation, methylcarbonyl, ethylcarbonyl, propylcarbonyl (e.g., n-propylcarbonyl and isopropylcarbonyl), butylcarbonyl (e.g., n-butylcarbonyl and tert-butylcarbonyl), and the like.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “carbamyl” to a group of formula —C(O)NH₂.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “thio” refers to a group of formula —SH.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfinyl” refers to a group of formula —S(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “carbonyl”, employed alone or in combination with other terms, refers to a —C(═O)— group, which may also be written as C(O).

As used herein, the term “carboxy” refers to a —C(O)OH group. In some embodiments, the “carboxy” group also refers to a bioisostere replacement group selected from the group consisting of:

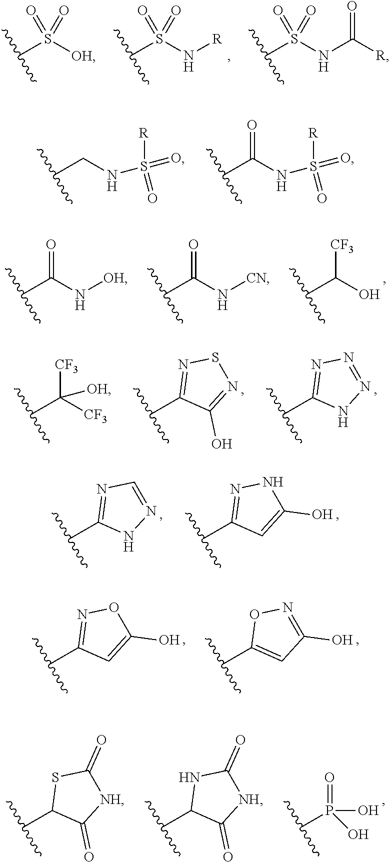

and the like, where R refers to a hydrogen, (C₁-C₈) alkyl, or C₆ aryl.

As used herein, the term “cyano-C₁₋₃ alkyl” refers to a group of formula —(C₁₋₃ alkylene)-CN.

As used herein, the term “HO—C₁₋₃ alkyl” refers to a group of formula —(C₁₋₃ alkylene)-OH.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which can be monocyclic or polycyclic (e.g., having 2, 3, or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups can have from 6 to 10 carbon atoms. In some embodiments, the aryl group is phenyl or naphthyl.

As used herein, “cycloalkyl” refers to non-aromatic cyclic hydrocarbons including cyclized alkyl and/or alkenyl groups. Cycloalkyl groups can include mono- or polycyclic (e.g., having 2, 3, or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. Example cycloalkyl groups include, without limitation, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur, and oxygen. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from nitrogen, sulfur, and oxygen. In some embodiments, the heteroaryl is a 5-6 monocyclic heteroaryl having 1 or 2 heteroatom ring members independently selected from nitrogen, sulfur, and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls include, without limitation, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls include, without limitation, pyridyl, pyrazinyl, pyrimidinyl, triazinyl, and pyridazinyl. Ring-forming carbon atoms of a heteroaryl group can be optionally substituted by 1 or 2 independently selected oxo or sulfide groups (e.g., C(O) or C(S)).

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9-, or 10-membered heterocycloalkyl groups. Heterocycloalkyl groups can also include spirocycles. Example heterocycloalkyl groups include, without limitation, pyrrolidin-2-one, 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring can be attached at any position of the ring, whereas a pyridin-3-yl ring is attached at the 3-position.

As used herein, the term “oxo” refers to an oxygen atom as a divalent substituent, forming a carbonyl group when attached to a carbon (e.g., C═O), or attached to a heteroatom forming a sulfoxide or sulfone group.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds provided herein that contain asymmetrically substituted carbon atoms can be isolated in optically active or racemic forms. Any appropriate method can be used to prepare optically active forms from, for example, optically inactive starting materials. For example, techniques such as resolution of racemic mixtures or stereoselective synthesis can be used to prepare optically active forms of a compound provided herein. Many geometric isomers of olefins, C═N double bonds, N═N double bonds, and the like also can be present in a compound described herein, and all such stable isomers are contemplated herein. Cis and trans geometric isomers of the compounds provided herein are described and can be isolated as a mixture of isomers or as separated isomeric forms. In some embodiments, a compound provided herein has the (R)-configuration. In some embodiments, a compound provided herein has the (S)-configuration.

Compounds provided herein also include tautomeric forms. Tautomeric forms result from the swapping of a single bond with an adjacent double bond together with the concomitant migration of a proton. Tautomeric forms include prototropic tautomers that are isomeric protonation states having the same empirical formula and total charge. Example prototropic tautomers include, without limitation, ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H-, and 4H-1,2,4-triazole, 1H- and 2H-isoindole, and 1H- and 2H-pyrazole. Tautomeric forms can be in equilibrium or sterically locked into one form by appropriate substitution. For example, in aqueous solution, pyrazoles can exhibit the following isomeric forms, which are referred to as tautomers of each other:

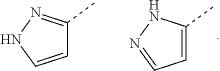

As readily understood by one skilled in the art, a wide variety of functional groups and other structures can exhibit tautomerism, and all tautomers of compounds as described herein are within the scope provided herein.

As used herein, the term “cell” is meant to refer to a cell that is in vitro, ex vivo, or in vivo. In some embodiments, an ex vivo cell can be part of a tissue sample excised from an organism such as a mammal (e.g., a human). In some embodiments, an in vitro cell can be a cell in cell culture. In some embodiments, an in vivo cell is a cell living in an organism such as a mammal (e.g., a human).

As used herein, the term “contacting” refers to the bringing together of indicated moieties or items in an in vitro system, an ex vivo system, or an in vivo system. For example, “contacting” a cell with a compound provided herein includes the act of administering that compound to a mammal (e.g., a human) containing that cell as well as, for example, introducing that compound into a cell culture containing that cell.

As used herein, the term “mammal” includes, without limitation, mice, rats, other rodents, rabbits, dogs, cats, swine, cattle, sheep, horses, elephants, deer, non human primates (e.g., monkeys and apes), house pets, and humans.

As used herein, the phrase “effective amount” or “therapeutically effective amount” refers to the amount of active compound or pharmaceutical agent that elicits the biological or medicinal response in a tissue, system, mammal, or human that is being sought by a researcher, veterinarian, medical doctor, or other clinician.

As used herein, the term “treating” or “treatment” refers to (a) inhibiting a disease, disorder, or condition, for example, inhibiting a disease, disorder, or condition in a mammal (e.g., human) that is experiencing or displaying the pathology or symptomatology of the disease, disorder, or condition (e.g., arresting further development of the pathology and/or symptomatology), or (b) ameliorating the disease, disorder, or condition, for example, ameliorating a disease, disorder, or condition in a mammal (e.g., a human) that is experiencing or displaying the pathology or symptomatology of the disease, disorder, or condition (e.g., reversing the pathology and/or symptomatology).

As used herein, the term “preventing” or “prevention” of a disease, disorder, or condition refers to decreasing the risk of occurrence of the disease, disorder, or condition in a mammal or group of mammals (e.g., a mammal or group of mammals predisposed to or susceptible to the disease, disorder, or condition). In some embodiments, preventing a disease, disorder, or condition refers to decreasing the possibility of acquiring the disease, disorder, or condition and/or its associated symptoms. In some embodiments, preventing a disease, disorder, or condition refers to completely or almost completely stopping the disease, disorder, or condition from occurring.

### Examples

### Assay for Total NADH and NAD⁺ Measurements in Cells

2,000 cells/well and, at the same time, test compounds dissolved in media at the desired concentration were added to each well. The treated cells were incubated overnight in CO₂ incubator. The cells were washed 6 times with PBS with 1 mM CaCl₂ and 1 mM MgCl₂. In the last wash, 10 μL of PBS with 1 mM CaCl₂ and 1 mM MgCl₂ per well were left in each well. Immediately thereafter, 10 μL 0.4% dodecyltrimethyl-ammonium bromide (DTAB) in phosphate buffer pH 8.0 as added, and the plates were shaked at 1000 rpm for 5 minutes. 60 μL reaction mix was added, and fluorescence at Ex/Em 530/585 was immediately monitored. A linear increase should be achieved with 8 cycles every 92 seconds, 10 number of flashes/well/cycle. The reaction mix was prepared per the following table, respecting the amount and order in which each component was added:

| | | | | | Mix for 1 | |
|---|---|---|---|---|---|---|
| Component | [Stock] | | [final] | | plate | |
| Sodium phosphate buffer | 100 | mM | | | 24473.3 | μL |
| (pH 8.0) | | | | | | |
| Ethanol | 95% | | 0.76% | | 266.7 | μL |
| Riboflavin 5′-monophosphate | 10 | mM | 4 | μM | 13.33 | μL |
| sodium salt hydrate (FMN) | | | | | | |
| Alcohol dehydrogenase | 3,400 | U/mL | 5.44 | U/mL | 53.3 | μL |
| (ADH) | | | | | | |
| Diaphorase | 100 | U/mL | 0.18 | U/mL | 60 | μL |
| Resazurin | 2 | mM | 8 | μM | 133.3 | μL |

| | | |
|---|---|---|
| Total: | 25 | mL |

In each table header, column “A” shows the increase in NAD⁺ levels in cells in response to the indicated test compound, as the percent of NAD⁺ levels compared to that in the absence of the test compound. A percentage of 100 percent would indicate no increase or decrease in NAD⁺ levels as compared to that measured for untreated control cells, while a percentage of 125 percent would indicate an increase in NAD⁺ levels of 25 percent as compared to that measured for untreated control cells.

TABLE 1

| BC No. | Structure | A |
|---|---|---|
| BC19118 | 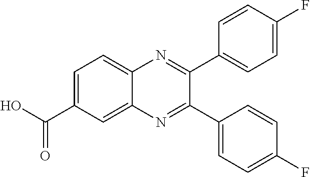 [SMILES: O=C(O)c1ccc2nc(-c3ccc(F)cc3)c(-c3ccc(F)cc3)nc2c1] | 108 |
| BC19500 | 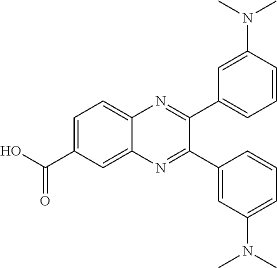 [SMILES: CN(C)c1cccc(-c2nc3ccc(C(=O)O)cc3nc2-c2cccc(N(C)C)c2)c1] | 107 |
| BC19499 | 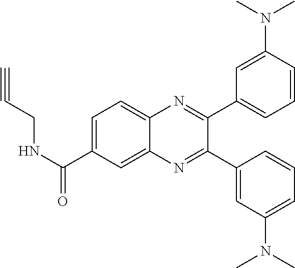 [SMILES: C#CCNC(=O)c1ccc2nc(-c3cccc(N(C)C)c3)c(-c3cccc(N(C)C)c3)nc2c1] | 165 |
| BC19502 | 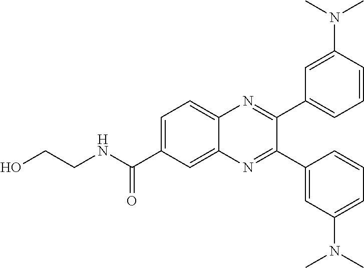 [SMILES: CN(C)c1cccc(-c2nc3ccc(C(=O)NCCO)cc3nc2-c2cccc(N(C)C)c2)c1] | 115 |
| BC19446 | 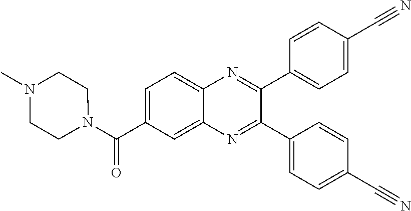 [SMILES: CN1CCN(C(=O)c2ccc3nc(-c4ccc(C#N)cc4)c(-c4ccc(C#N)cc4)nc3c2)CC1] | 106 |
| BC20287 | 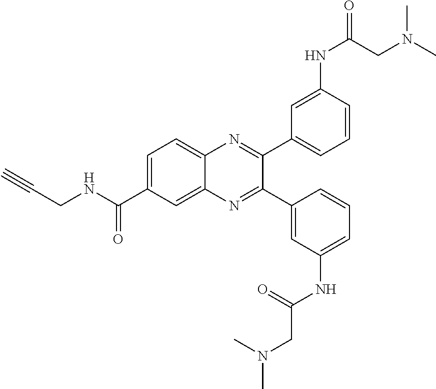 [SMILES: C#CCNC(=O)c1ccc2nc(-c3cccc(NC(=O)CN(C)C)c3)c(-c3cccc(NC(=O)CN(C)C)c3)nc2c1] | 120 |

TABLE 2

| BC No. | Structure | A |
|---|---|---|
| BC19109 | 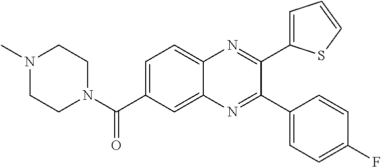 [SMILES: CN1CCN(C(=O)c2ccc3nc(-c4cccs4)c(-c4ccc(F)cc4)nc3c2)CC1] | 111 |
| BC19119 | 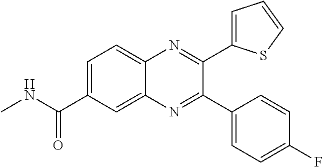 [SMILES: CNC(=O)c1ccc2nc(-c3cccs3)c(-c3ccc(F)cc3)nc2c1] | 112 |
| BC19187 | 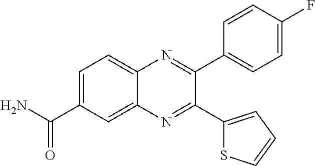 [SMILES: NC(=O)c1ccc2nc(-c3ccc(F)cc3)c(-c3cccs3)nc2c1] | 113 |
| BC19189 | 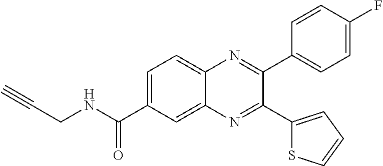 [SMILES: C#CCNC(=O)c1ccc2nc(-c3ccc(F)cc3)c(-c3cccs3)nc2c1] | 109 |
| BC19156 | 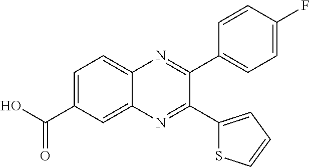 [SMILES: O=C(O)c1ccc2nc(-c3ccc(F)cc3)c(-c3cccs3)nc2c1] | 105 |

TABLE 3a

| BC No. | Structure | A |
|---|---|---|
| BC19144 | 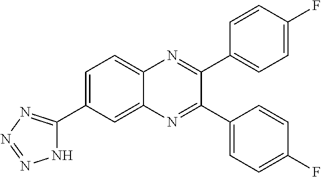 [SMILES: Fc1ccc(-c2nc3ccc(-c4nnn[nH]4)cc3nc2-c2ccc(F)cc2)cc1] | 107 |
| BC19145 | 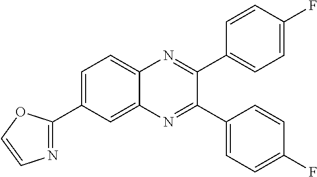 [SMILES: Fc1ccc(-c2nc3ccc(-c4ncco4)cc3nc2-c2ccc(F)cc2)cc1] | 106 |
| BC19193 | 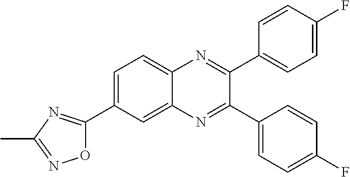 [SMILES: Cc1noc(-c2ccc3nc(-c4ccc(F)cc4)c(-c4ccc(F)cc4)nc3c2)n1] | 109 |
| BC19113 | 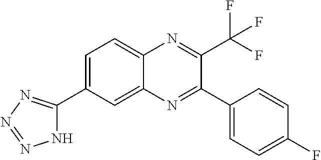 [SMILES: Fc1ccc(-c2nc3cc(-c4nnn[nH]4)ccc3nc2C(F)(F)F)cc1] | 110 |
| BC19186 | 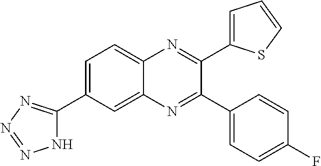 [SMILES: Fc1ccc(-c2nc3cc(-c4nnn[nH]4)ccc3nc2-c2cccs2)cc1] | 105 |
| BC19248 | 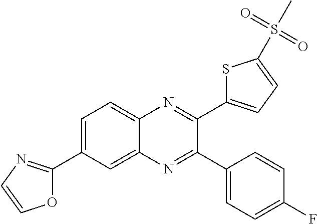 [SMILES: CS(=O)(=O)c1ccc(-c2nc3ccc(-c4ncco4)cc3nc2-c2ccc(F)cc2)s1] | 125 |
| BC19249 | 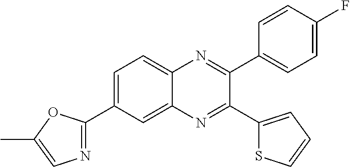 [SMILES: Cc1cnc(-c2ccc3nc(-c4ccc(F)cc4)c(-c4cccs4)nc3c2)o1] | 119 |
| BC19444 | 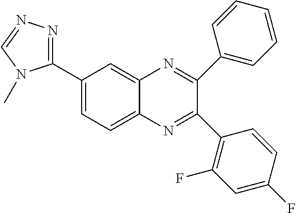 [SMILES: Cn1cnnc1-c1ccc2nc(-c3ccc(F)cc3F)c(-c3ccccc3)nc2c1] | 112 |

TABLE 3b

| BC | ZE | | |
|---|---|---|---|
| No. | No. | Structure | A |
| | ZE18- 0075 | 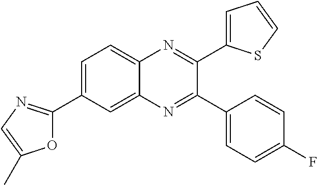 [SMILES: Cc1cnc(-c2ccc3nc(-c4cccs4)c(-c4ccc(F)cc4)nc3c2)o1] | |
| | ZE18- 0591 | 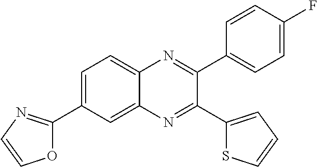 [SMILES: Fc1ccc(-c2nc3ccc(-c4ncco4)cc3nc2-c2cccs2)cc1] | |
| | ZE18- 0592 | 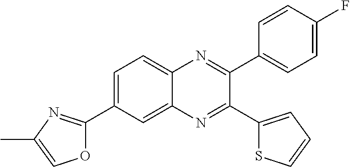 [SMILES: Cc1coc(-c2ccc3nc(-c4ccc(F)cc4)c(-c4cccs4)nc3c2)n1] | |
| | ZE18- 0593 | 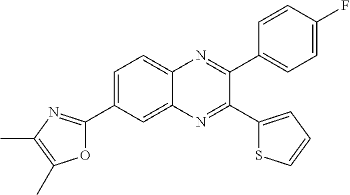 [SMILES: Cc1nc(-c2ccc3nc(-c4ccc(F)cc4)c(-c4cccs4)nc3c2)oc1C] | |
| | ZE18- 0594 | 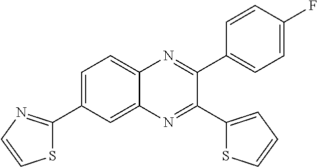 [SMILES: Fc1ccc(-c2nc3ccc(-c4nccs4)cc3nc2-c2cccs2)cc1] | |
| | ZE18- 0595 | 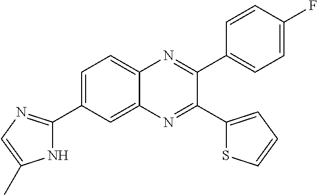 [SMILES: Cc1cnc(-c2ccc3nc(-c4ccc(F)cc4)c(-c4cccs4)nc3c2)[nH]1] | |
| | ZE18- 0596 | 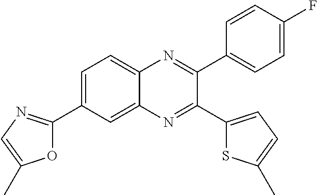 [SMILES: Cc1cnc(-c2ccc3nc(-c4ccc(F)cc4)c(-c4ccc(C)s4)nc3c2)o1] | |
| | ZE18- 0597 | 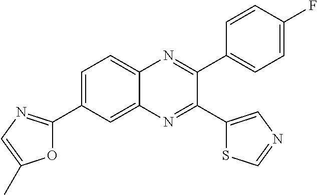 [SMILES: Cc1cnc(-c2ccc3nc(-c4ccc(F)cc4)c(-c4cncs4)nc3c2)o1] | |
| | ZE18- 0598 | 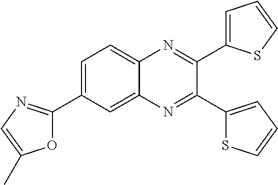 [SMILES: Cc1cnc(-c2ccc3nc(-c4cccs4)c(-c4cccs4)nc3c2)o1] | |
| | ZE18- 0600 | 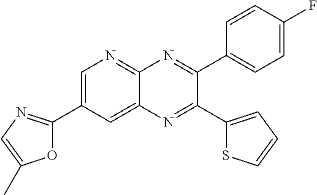 [SMILES: Cc1cnc(-c2cnc3nc(-c4ccc(F)cc4)c(-c4cccs4)nc3c2)o1] | |
| | ZE18- 0601 | 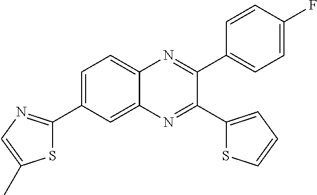 [SMILES: Cc1cnc(-c2ccc3nc(-c4ccc(F)cc4)c(-c4cccs4)nc3c2)s1] | |
| | ZE18- 0599 | 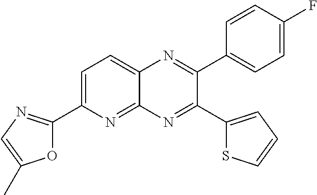 [SMILES: Cc1cnc(-c2ccc3nc(-c4ccc(F)cc4)c(-c4cccs4)nc3n2)o1] | |

TABLE 4

| BC No. | Structure | A |
|---|---|---|
| BC19105 | 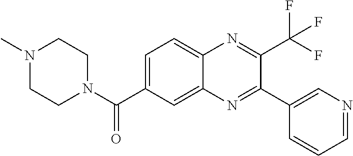 [SMILES: CN1CCN(C(=O)c2ccc3nc(C(F)(F)F)c(-c4cccnc4)nc3c2)CC1] | 105 |
| BC19149 | 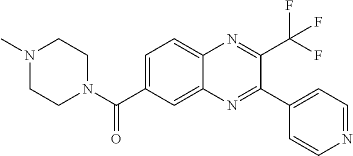 [SMILES: CN1CCN(C(=O)c2ccc3nc(C(F)(F)F)c(-c4ccncc4)nc3c2)CC1] | 106 |
| BC19110 | 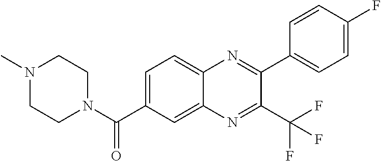 [SMILES: CN1CCN(C(=O)c2ccc3nc(-c4ccc(F)cc4)c(C(F)(F)F)nc3c2)CC1] | 105 |
| BC19117 | 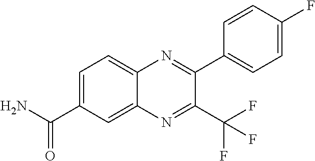 [SMILES: NC(=O)c1ccc2nc(-c3ccc(F)cc3)c(C(F)(F)F)nc2c1] | 116 |

TABLE 5

| | ZE | | |
|---|---|---|---|
| BC No. | No. | Structure | A |
| BC20200 | | 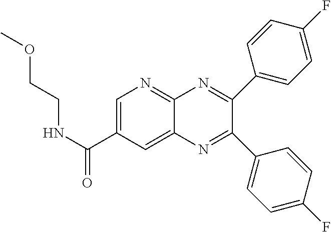 [SMILES: COCCNC(=O)c1cnc2nc(-c3ccc(F)cc3)c(-c3ccc(F)cc3)nc2c1] | 132 |
| BC20201 | | 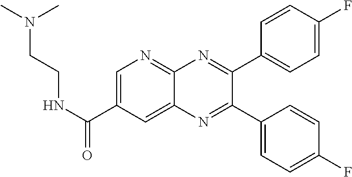 [SMILES: CN(C)CCNC(=O)c1cnc2nc(-c3ccc(F)cc3)c(-c3ccc(F)cc3)nc2c1] | 133 |
| BC20257 | | 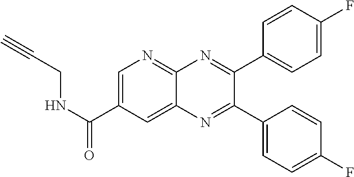 [SMILES: C#CCNC(=O)c1cnc2nc(-c3ccc(F)cc3)c(-c3ccc(F)cc3)nc2c1] | 130 |
| | ZE18- 0572 | 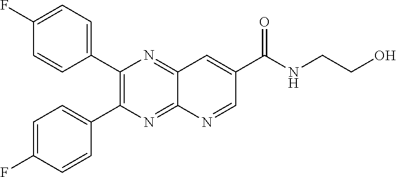 [SMILES: O=C(NCCO)c1cnc2nc(-c3ccc(F)cc3)c(-c3ccc(F)cc3)nc2c1] | |
| | ZE18- 0573 | 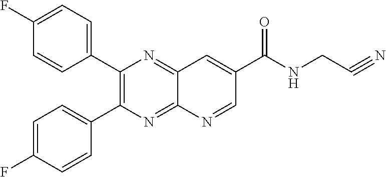 [SMILES: N#CCNC(=O)c1cnc2nc(-c3ccc(F)cc3)c(-c3ccc(F)cc3)nc2c1] | |
| | ZE18- 0574 | 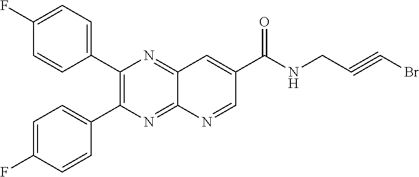 [SMILES: O=C(NCC#CBr)c1cnc2nc(-c3ccc(F)cc3)c(-c3ccc(F)cc3)nc2c1] | |
| | ZE18- 0575 | 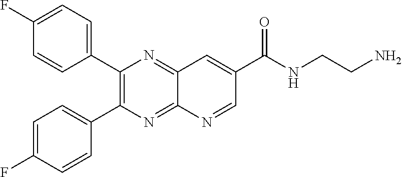 [SMILES: NCCNC(=O)c1cnc2nc(-c3ccc(F)cc3)c(-c3ccc(F)cc3)nc2c1] | |
| | ZE18- 0576 | 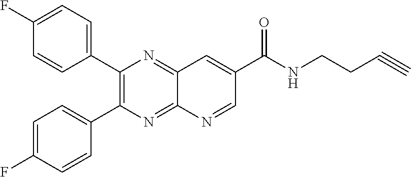 [SMILES: C#CCCNC(=O)c1cnc2nc(-c3ccc(F)cc3)c(-c3ccc(F)cc3)nc2c1] | |
| | ZE18- 0577 | 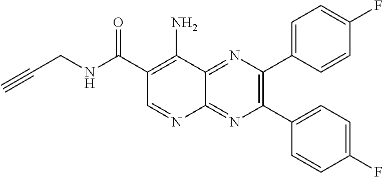 [SMILES: C#CCNC(=O)c1cnc2nc(-c3ccc(F)cc3)c(-c3ccc(F)cc3)nc2c1N] | |
| | ZE18- 0579 | 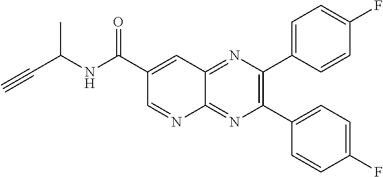 [SMILES: C#CC(C)NC(=O)c1cnc2nc(-c3ccc(F)cc3)c(-c3ccc(F)cc3)nc2c1] | |
| | ZE18- 0580 | 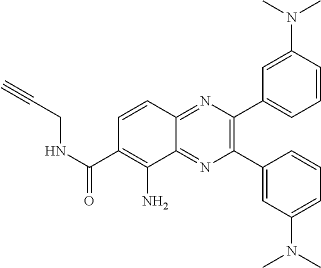 [SMILES: C#CCNC(=O)c1ccc2nc(-c3cccc(N(C)C)c3)c(-c3cccc(N(C)C)c3)nc2c1N] | |
| | ZE18- 0583 | 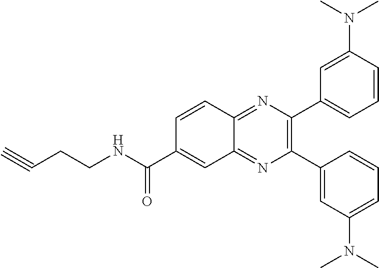 [SMILES: C#CCCNC(=O)c1ccc2nc(-c3cccc(N(C)C)c3)c(-c3cccc(N(C)C)c3)nc2c1] | |
| | ZE18- 0584 | 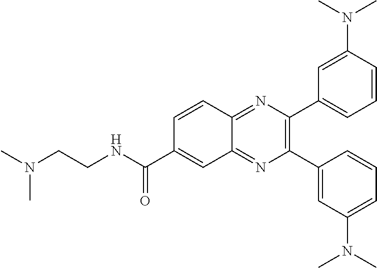 | |
| | ZE18- 0585 | 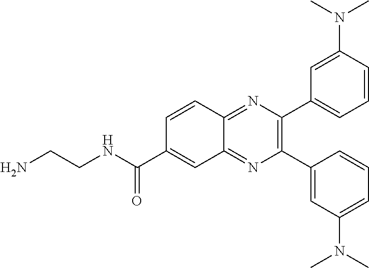 [SMILES: CN(C)c1cccc(-c2nc3ccc(C(=O)NCCN)cc3nc2-c2cccc(N(C)C)c2)c1] | |
| | ZE18- 0587 | 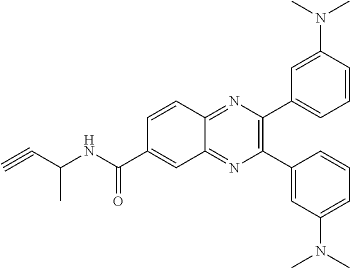 [SMILES: C#CC(C)NC(=O)c1ccc2nc(-c3cccc(N(C)C)c3)c(-c3cccc(N(C)C)c3)nc2c1] | |
| | ZE18- 0589 | 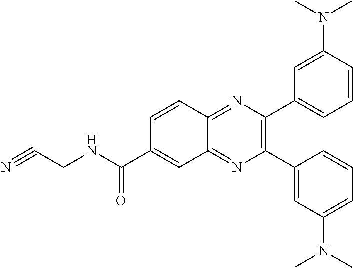 [SMILES: CN(C)c1cccc(-c2nc3ccc(C(=O)NCC#N)cc3nc2-c2cccc(N(C)C)c2)c1] | |
| | ZE18- 0590 | 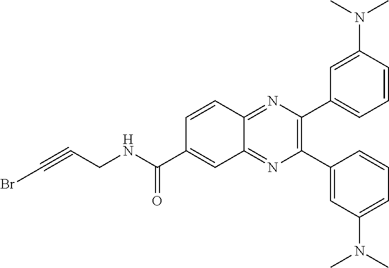 [SMILES: CN(C)c1cccc(-c2nc3ccc(C(=O)NCC#CBr)cc3nc2-c2cccc(N(C)C)c2)c1] | |

TABLE 6

| BC No. | ZE No. | Structure | A |
|---|---|---|---|
| | ZE18-0568 | 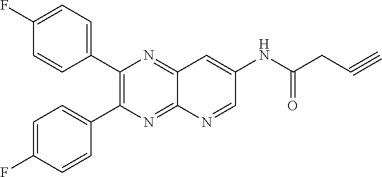 [SMILES: C#CCC(=O)Nc1cnc2nc(-c3ccc(F)cc3)c(-c3ccc(F)cc3)nc2c1] | |
| | ZE18-0569 | 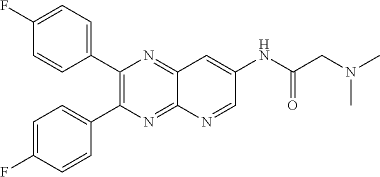 [SMILES: CN(C)CC(=O)Nc1cnc2nc(-c3ccc(F)cc3)c(-c3ccc(F)cc3)nc2c1] | |
| | ZE18-0570 | 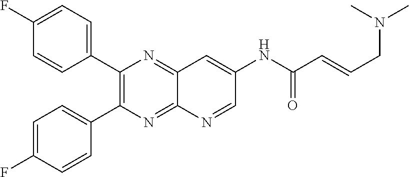 [SMILES: CN(C)C/C=C/C(=O)Nc1cnc2nc(-c3ccc(F)cc3)c(-c3ccc(F)cc3)nc2c1] | |
| | ZE18-0571 | 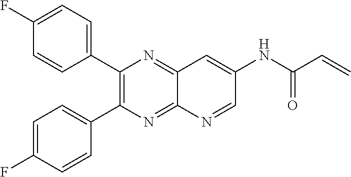 [SMILES: C=CC(=O)Nc1cnc2nc(-c3ccc(F)cc3)c(-c3ccc(F)cc3)nc2c1] | |
| | ZE18-0581 | 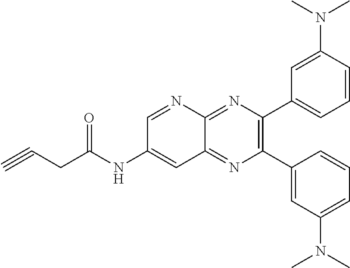 [SMILES: C#CCC(=O)Nc1cnc2nc(-c3cccc(N(C)C)c3)c(-c3cccc(N(C)C)c3)nc2c1] | |
| | ZE18-0586 | 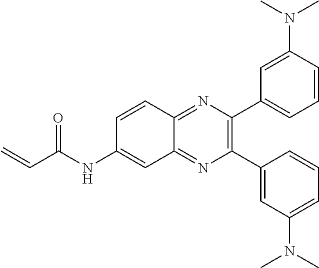 [SMILES: C=CC(=O)Nc1ccc2nc(-c3cccc(N(C)C)c3)c(-c3cccc(N(C)C)c3)nc2c1] | |
| | ZE18-0588 | 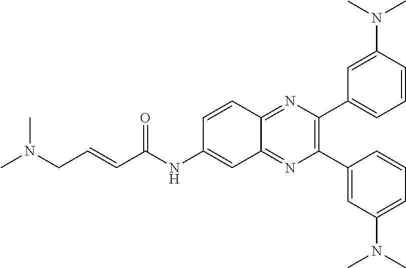 | |

TABLE 7

| BC No. | ZE No. | Structure | A |
|---|---|---|---|
| | ZE18-0578 | 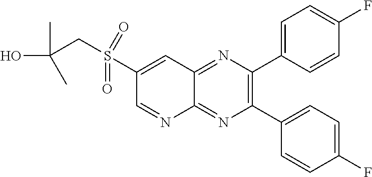 [SMILES: CC(C)(O)CS(=O)(=O)c1cnc2nc(-c3ccc(F)cc3)c(-c3ccc(F)cc3)nc2c1] | |
| | ZE18-0582 | 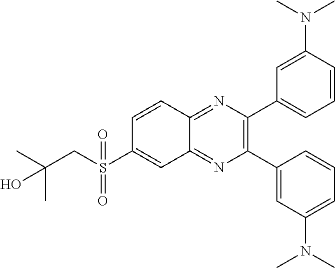 [SMILES: CN(C)c1cccc(-c2nc3ccc(S(=O)(=O)CC(C)(C)O)cc3nc2-c2cccc(N(C)C)c2)c1] | |

### Numbered Paragraphs

1. A method for increasing NAMPT activity within a mammal, wherein said method comprises administering, to said mammal, an effective amount of a compound of Formula (I):

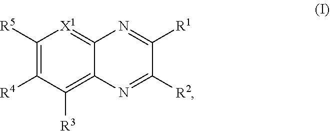

or a pharmaceutically acceptable salt thereof, wherein:
X¹ is selected from N and CR⁶;
R³, R⁴, R⁵, and R⁶ are each independently selected from H, Cy¹, halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, C(NR^{e1})NR^{c1}R^{d1}, C(NR^{e1})NR^{c1}OR^{a1}, NR^{c1}C(O)NR^{c1}R^{d1}, NR^{c1}C(S)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from R⁷;
each R⁷ is independently selected from Cy¹, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
each Cy¹ is independently selected from C₆₋₁₀ aryl, C₃₋₁₀ cycloalkyl, 5-10 membered heteroaryl, and 4-10 membered heterocycloalkyl, each of which is optionally substituted with 1, 2, 3, 4, or 5 substituents independently selected from R^{Cy1};
each R^{Cy1} is independently selected from halo, CN, NO₂, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 5-10 membered heteroaryl, 4-10 membered heterocycloalkyl, OR^{a1}, C(O)R^{b1}, C(O)NR^{c1}R^{d1}, C(O)OR^{a1}NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}, wherein said C₁₋₆ alkyl, C₂₋₆ alkenyl, C₂₋₆ alkynyl, C₃₋₁₀ cycloalkyl, C₆₋₁₀ aryl, 5-10 membered heteroaryl, and 4-10 membered heterocycloalkyl are each optionally substituted with 1, 2, or 3 substituents independently selected from R⁸;
each R⁸ is independently selected from C₁₋₆ alkyl, C₂₋₆ alkenyl, C₂₋₆ alkynyl, C₃₋₁₀ cycloalkyl, C₆₋₁₀ aryl, 5-10 membered heteroaryl, 4-10 membered heterocycloalkyl, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}, wherein said C₁₋₆ alkyl, C₂₋₆ alkenyl, C₂₋₆ alkynyl, C₃₋₁₀ cycloalkyl, C₆₋₁₀ aryl, 5-10 membered heteroaryl, and 4-10 membered heterocycloalkyl are each optionally substituted with 1, 2, 3, 4, or 5 substituents independently selected from R^{g};
R¹ and R² are each independently selected from R⁹ and S(O)₂R⁹;
each R⁹ is independently selected from C₁₋₆ haloalkyl, C₆₋₁₀ aryl, 5-10 membered heteroaryl, and 4-10 membered heterocycloalkyl, each of which is optionally substituted with 1, 2, 3, 4, or 5 substituents independently selected from R¹⁰;
each R¹⁰ is independently selected from halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from R¹¹;
each R¹¹ is independently selected from CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
each R^{e1} is selected from H, OR^{a1} NR^{c1}R^{d1} and C₁₋₄ haloalkyl;
each R^{a1}, R^{b1}, R^{c1} and R^{d1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, 3, 4, or 5 substituents independently selected from R^{g};
or any R^{c1} and R^{d1} together with the N atom to which they are attached form a 4-7 membered heterocycloalkyl, which is optionally substituted with 1, 2, or 3 substituents independently selected from R^{g}; and
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkenylcarbonyl, C₁₋₆ alkynylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, and any C₁₋₆ alkyl, C₁₋₆ alkoxy, C₆₋₁₀ aryl, C₃₋₁₀ cycloalkyl, 5-10 membered heteroaryl, or 4-10 membered heterocycloalkyl of R^{g} is optionally substituted with 1, 2, or 3 substituents independently selected from OH, NO₂, CN, halo, C₁₋₆ alkyl, C₁₋₄ haloalkyl, C₁₋₆ alkoxy, and C₁₋₆ haloalkoxy.
2. The method of paragraph 1, wherein the compound of Formula (I) has formula:

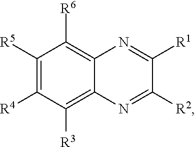

or a pharmaceutically acceptable salt thereof.
3. The method of paragraph 2, wherein:
   R³, R⁵, and R⁶ are each independently selected from H, halo, C₁₋₆ alkyl, C₁₋₆ haloalkyl, OR^{a1}, C(O)OR^{a1} and NR^{c1}R^{d1} wherein said C₁₋₆ alkyl is optionally substituted with a substituent selected from OR^{a1}, C(O)R^{b1}, C(O)NR^{c1}R^{d1}, C(O)OR^{a1}, NR^{c1}R^{d1}, S(O)₂R^{b1}, and S(O)₂NR^{c1}R^{d1}; and
   R⁴ is selected from Cy¹, C(O)NR^{c1}R^{d1}, C(O)OR^{a1}, NR^{c1}R^{d1}, NR^{c1}C(O)NR^{c1}R^{d1}, NR^{c1}C(S)NR^{c1}R^{d1}, and S(O)₂NR^{c1}R^{d1}.
4. The method of paragraph 3, wherein:
   R³, R⁵, and R⁶ are each independently selected from H, halo, C₁₋₆ alkyl, C₁₋₆ haloalkyl, C₁₋₆ alkoxy, C₁₋₆ haloalkoxy; and
   R⁴ is selected from Cy¹, C(O)NR^{c1}R^{d1}, and C(O)OR^{a1}.
5. The method of any one of paragraphs 1-4, wherein the compound of Formula (I) has formula:

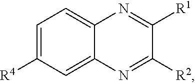

or a pharmaceutically acceptable salt thereof.
6. The method of paragraph 1, wherein the compound of Formula (I) has formula:

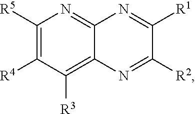

or a pharmaceutically acceptable salt thereof.
7. The method of paragraph 6, wherein:
   R³ and R⁵ are each independently selected from H, halo, C₁₋₆ alkyl, C₁₋₆ haloalkyl, OR^{a1}, C(O)OR^{a1}, and NR^{c1}R^{d1}, wherein said C₁₋₆ alkyl is optionally substituted with a substituent selected from OR^{a1}, C(O)R^{b1}, C(O)NR^{c1}R^{d1}, C(O)OR^{a1}, NR^{c1}R^{d1}, S(O)₂R^{b1}, and S(O)₂NR^{c1}R^{d1}; and
   R⁴ is selected from Cy¹, C(O)NR^{c1}R^{d1}, C(O)OR^{a1}, NR^{c1}R^{d1}, NR^{c1}C(O)NR^{c1}R^{d1}, NR^{c1}C(S)NR^{c1}R^{d1}, and S(O)₂NR^{c1}R^{d1}.
8. The method of paragraph 7, wherein:
   R³ and R⁵ are each independently selected from H, halo, C₁₋₆ alkyl, C₁₋₆ haloalkyl, C₁₋₆ alkoxy, C₁₋₆ haloalkoxy; and
   R⁴ is selected from Cy¹, C(O)NR^{c1}R^{d1} and C(O)OR^{a1}.
9. The method of any one of paragraphs 1 and 6-8, wherein the compound of Formula (I) has formula:

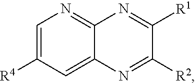

or a pharmaceutically acceptable salt thereof.
10. The method of any one of paragraphs 1-9, wherein R⁴ is Cy¹.
11. The method of any one of paragraphs 1-9, wherein R⁴ is C(O)NR^{c1}R^{d1}.
12. The method of any one of paragraphs 1-9, wherein R⁴ is C(O)OR^{a1}.
13. The method of paragraph 1, wherein the compound of Formula (I) has formula:

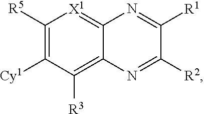

or a pharmaceutically acceptable salt thereof.
14. The method of paragraph 1, wherein the compound of Formula (I) has formula:

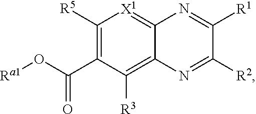

or a pharmaceutically acceptable salt thereof.
15. The method of paragraph 1, wherein the compound of Formula (I) has formula:

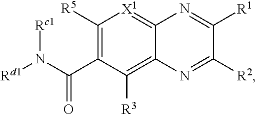

or a pharmaceutically acceptable salt thereof.
16. The method of paragraph 15, wherein the compound of Formula (I) has formula:

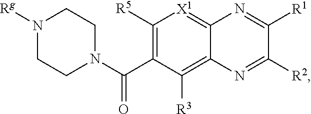

or a pharmaceutically acceptable salt thereof.
17. The method of any one of paragraphs 13-16, wherein R³, R⁵, and R⁶ are each independently selected from H, halo, C₁₋₆ alkyl, C₁₋₆ haloalkyl, OR^{a1}, C(O)OR^{a1}, and NR^{c1}R^{d1}, wherein said C₁₋₆ alkyl is optionally substituted with a substituent selected from OR^{a1}, C(O)R^{b1}, C(O)NR^{c1}R^{d1}, C(O)OR^{a1} NR^{c1}R^{d1}, S(O)₂R^{b1}, and S(O)₂NR^{c1}R^{d1}.
18. The method of any one of paragraphs 13-16, wherein R³, R⁵, and R⁶ are each independently selected from H, halo, C₁₋₆ alkyl, C₁₋₆ haloalkyl, C₁₋₆ alkoxy, and C₁₋₆ haloalkoxy.
19. The method of any one of paragraphs 13-16, wherein R³, R⁵, and R⁶ are each H.
20. The method of any one of paragraphs 1-15, wherein R^{c1} and R^{d1} are each independently selected from H, C₁₋₆ alkyl, C₁₋₄ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with a substituent selected from OH, thio, C₁₋₆ alkoxy, C₁₋₆ thialkoxy, C₁₋₆ haloalkoxy, amino, C₁₋₆ alkylamino, and di(C₁₋₆ alkyl)amino.
21. The method of paragraph 20, wherein R^{c1} and R^{d1} are each independently selected from H, C₁₋₆ alkyl, and C₂₋₆ alkynyl, wherein said C₁₋₆ alkyl is optionally substituted with a substituent selected from OH, C₁₋₆ alkoxy, and di(C₁₋₆ alkyl)amino.
22. The method of any one of paragraph 1-15, wherein R^{c1} and R^{d1} together with the N atom to which they are attached form a 4-7 membered heterocycloalkyl, which is optionally substituted with 1 or 2 substituents independently selected from R^{g}.
23. The method of paragraph 22, wherein R^{c1} and R^{d1} together with the N atom to which they are attached form a ring selected from morpholinyl, thiomorpholinyl, piperazinyl, piperidinyl, pyrrolidinyl, and azepanyl, each of which is optionally substituted with 1 or 2 substituents independently selected from R^{g}.
24. The method of paragraph 23, wherein R^{c1} and R^{d1} together with the N atom to which they are attached form a piperazinyl ring of formula:

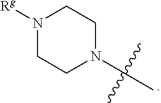

25. The method of any one of paragraphs 1-24, wherein R^{a1} is selected from H, C₁₋₆ alkyl, C₁₋₄ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with a substituent selected from OH, thio, C₁₋₆ alkoxy, C₁₋₆ thialkoxy, C₁₋₆ haloalkoxy, amino, C₁₋₆ alkylamino, and di(C₁₋₆ alkyl)amino.
26. The method of paragraph 25, wherein R^{a1} is selected from H and C₁₋₆ alkyl.
27. The method of any one of paragraphs 1-26, wherein Cy¹ is 5-10 membered heteroaryl, which is optionally substituted with 1 or 2 independently selected R^{Cy1}.
28. The method of paragraph 27, wherein Cy¹ is selected from 1,2,4-triazolyl, tetrazolyl, oxazolyl, isoxazolyl, thiophenyl, indolyl, pyrimidinyl, pyrrolopyridinyl, benzoxadiazolyl, 1,3,4-oxadiazolyl, 1,2,3-triazolyl, 1,2,4-oxadiazolyl, thiazolyl, pyridinyl, benzoxazinyl, pyrazolyl, and indazolyl, each of which is optionally substituted with 1 or 2 substituents independently selected from R^{Cy1}.
29. The method of paragraph 27, wherein Cy¹ is selected from 1,2,4-triazolyl, tetrazolyl, oxazolyl, and 1,2,4-oxadiazolyl, each of which is optionally substituted with 1 or 2 substituents independently selected from R^{Cy1}.
30. The method of any one of paragraphs 1-29, wherein R^{Cy1} is selected from C₁₋₆ alkyl, C₁₋₆ haloalkyl, C₂₋₆ alkenyl, and C₂₋₆ alkynyl, each of which is optionally substituted with 1 or 2 independently selected R⁸.
31. The method of paragraph 30, wherein R^{Cy1} is C₁₋₆ alkyl, optionally substituted with R⁸.
32. The method of any one of paragraphs 1-31, wherein R⁸ is selected from OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}.
33. The method of paragraph 32, wherein R⁸ is selected from OH, thio, C₁₋₆ alkoxy, C₁₋₆ thialkoxy, C₁₋₆ haloalkoxy, amino, C₁₋₆ alkylamino, and di(C₁₋₆ alkyl)amino.
34. The method of any one of paragraphs 1-33, wherein R¹ and R² are each independently selected from C₁₋₆ haloalkyl, C₆₋₁₀ aryl, and 5-10 membered heteroaryl, wherein said C₆₋₁₀ aryl and 5-10 membered heteroaryl are each optionally substituted with 1 or 2 independently selected from R¹⁰.
35. The method of paragraph 34, wherein R¹ and R² are each independently a C₆₋₁₀ aryl, optionally substituted with 1 or 2 independently selected from R¹⁰.
36. The method of paragraph 34, wherein:
   R¹ is 5-10 membered heteroaryl, optionally substituted with 1 or 2 independently selected from R¹⁰; and
   R² is C₆₋₁₀ aryl, optionally substituted with 1 or 2 independently selected from R¹⁰.
37. The method of paragraph 34, wherein:
   R¹ is C₆₋₁₀ aryl, optionally substituted with 1 or 2 independently selected from R¹⁰; and
   R² is 5-10 membered heteroaryl, optionally substituted with 1 or 2 independently selected from R¹⁰.
38. The method of paragraph 34, wherein:
   R¹ is C₁₋₆ haloalkyl; and
   R² is C₆₋₁₀ aryl, optionally substituted with 1 or 2 independently selected from R¹⁰.
39. The method of paragraph 34, wherein:
   R¹ is C₁₋₆ haloalkyl; and
   R² is 5-10 membered heteroaryl, optionally substituted with 1 or 2 independently selected from R¹⁰.
40. The method of paragraph 34, wherein
   R¹ is C₆₋₁₀ aryl, optionally substituted with 1 or 2 independently selected from R¹⁰; and
   R² is C₁₋₆ haloalkyl.
41. The method of any one of paragraphs 1-40, wherein R¹⁰ is selected from halo, CN, C(O)NR^{c1}R^{d1}, C(O)OR^{a1}, NR^{c1}R^{d1}, NR^{c1}C(O)R^{b1}, NR^{c1}S(O)₂R^{b1}, S(O)₂R^{b1}, and S(O)₂NR^{c1}R^{d1}.
42. The method of paragraph 41, wherein R¹⁰ is selected from halo, CN, NR^{c1}R^{d1}, NR^{c1}C(O)R^{b1}, and S(O)₂R^{b1}.
43. The method of paragraph 1, wherein:
   R³, R⁵, and R⁶ are each independently selected from H, halo, C₁₋₆ alkyl, C₁₋₆ haloalkyl, OR^{a1}, C(O)OR^{a1}, and NR^{c1}R^{d1}, wherein said C₁₋₆ alkyl is optionally substituted with a substituent selected from OR^{a1}, C(O)R^{b1}, C(O)NR^{c1}R^{d1}, C(O)OR^{a1}, NR^{c1}R^{d1}, S(O)₂R^{b1}, and S(O)₂NR^{c1}R^{d1};
   R⁴ is selected from Cy¹, C(O)NR^{c1}R^{d1}, C(O)OR^{a1}, NR^{c1}R^{d1}, NR^{c1}C(O)NR^{c1}R^{d1}, NR^{c1}C(S)NR^{c1}R^{d1}, and S(O)₂NR^{c1}R^{d1};
   R^{c1} and R^{d1} are each independently selected from H, C₁₋₆ alkyl, C₁₋₄ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with a substituent selected from OH, thio, C₁₋₆ alkoxy, C₁₋₆ thialkoxy, C₁₋₆ haloalkoxy, amino, C₁₋₆ alkylamino, and di(C₁₋₆ alkyl)amino; or
   R^{c1} and R^{d1} together with the N atom to which they are attached form a 4-7 membered heterocycloalkyl, which is optionally substituted with 1 or 2 substituents independently selected from R^{g};
   R^{a1} is selected from H, C₁₋₆ alkyl, C₁₋₄ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with a substituent selected from OH, thio, C₁₋₆ alkoxy, C₁₋₆ thialkoxy, C₁₋₆ haloalkoxy, amino, C₁₋₆ alkylamino, and di(C₁₋₆ alkyl)amino;
   Cy¹ is 5-10 membered heteroaryl, which is optionally substituted with 1 or 2 independently selected R^{Cy1};
   R^{Cy1} is selected from C₁₋₆ alkyl, C₁₋₆ haloalkyl, C₂₋₆ alkenyl, and C₂₋₆ alkynyl, each of which is optionally substituted with 1 or 2 independently selected R⁸;
   R⁸ is selected from OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
   R¹ and R² are each independently selected from C₁₋₆ haloalkyl, C₆₋₁₀ aryl, and 5-10 membered heteroaryl, wherein said C₆₋₁₀ aryl and 5-10 membered heteroaryl are each optionally substituted with 1 or 2 independently selected from R¹⁰; and
   R¹⁰ is selected from halo, CN, C(O)NR^{c1}R^{d1}, C(O)OR^{a1}, NR^{c1}R^{d1}, NR^{c1}C(O)R^{b1}, NR^{c1}S(O)_{2R}^{b1}, S(O)₂R^{b1}, and S(O)₂NR^{c1}R^{d1}.
44. The method of paragraph 1, wherein:
   R³, R⁵, and R⁶ are each independently selected from H, halo, C₁₋₆ alkyl, C₁₋₆ haloalkyl, C₁₋₆ alkoxy, and C₁₋₆ haloalkoxy; and
   R⁴ is selected from Cy¹, C(O)NR^{c1}R^{d1}, and C(O)OR^{a1};
   R^{c1} and R^{d1} are each independently selected from H, C₁₋₆ alkyl, and C₂₋₆ alkynyl, wherein said C₁₋₆ alkyl is optionally substituted with a substituent selected from OH, C₁₋₆ alkoxy, and di(C₁₋₆ alkyl)amino; or
   R^{c1} and R^{d1} together with the N atom to which they are attached form piperazinyl, optionally substituted with 1 or 2 substituents independently selected from R^{g};
   R^{a1} is selected from H and C₁₋₆ alkyl;
   Cy¹ is selected from 1,2,4-triazolyl, tetrazolyl, oxazolyl, isoxazolyl, thiophenyl, indolyl, pyrimidinyl, pyrrolopyridinyl, benzoxadiazolyl, 1,3,4-oxadiazolyl, 1,2,3-triazolyl, 1,2,4-oxadiazolyl, thiazolyl, pyridinyl, benzoxazinyl, pyrazolyl, and indazolyl, each of which is optionally substituted with 1 or 2 substituents independently selected from R^{Cy1};
   R^{Cy1} is C₁₋₆ alkyl, optionally substituted with R⁸;
   R⁸ is selected from OH, thio, C₁₋₆ alkoxy, C₁₋₆ thialkoxy, C₁₋₆ haloalkoxy, amino, C₁₋₆ alkylamino, and di(C₁₋₆ alkyl)amino;
   R¹ and R² are each independently selected from C₁₋₆ haloalkyl, C₆₋₁₀ aryl, and 5-10 membered heteroaryl, wherein said C₆₋₁₀ aryl and 5-10 membered heteroaryl are each optionally substituted with 1 or 2 independently selected from R¹⁰; and
   R¹⁰ is selected from halo, CN, NR^{c1}R^{d1}, NR^{c1}C(O)R^{b1}, and S(O)₂R^{b1}.
45. The method of paragraph 1, wherein the compound of Formula (I) is selected from any one of the compounds listed in Table 1, Table 2, Table 3a, Table 3b, Table 4, Table 5, Table 6, or Table 7, or a pharmaceutically acceptable salt thereof.
46. A method of treating a mammal having a disease, disorder, or condition responsive to increasing NAMPT activity within the mammal, wherein said method comprises administering, to said mammal, a therapeutically effective amount of a compound of Formula (I) as recited in paragraphs 1-45, or a pharmaceutically acceptable salt thereof.
47. The method of paragraph 46, wherein said mammal has a traumatic nerve injury, a neuropathy, a neurodegenerative condition, a central demyelinating disorder, a peripheral demyelinating disorder, a primarily inflammatory neuropathy, glaucoma, an ischemic injury, a retinal or optic nerve ischemia, a stroke, age-related vision or hearing loss, hepatosteatosis, an insulin resistance syndrome, obesity, sarcopenia, a disorder of inflammation or auto-immunity, skin aging, a cardiovascular disease, heart failure, an acute or chronic kidney injury, or infertility.
48. A compound selected from BC20287 and any one of the compounds listed in Table 3b, Table 5, Table 6, or Table 7, or a pharmaceutically acceptable salt thereof.
49. A pharmaceutical composition comprising a compound of paragraph 48, or a pharmaceutically acceptable salt thereof, and a pharmaceutically acceptable carrier.
50. A method for increasing NAMPT activity within a mammal, wherein said method comprises administering, to said mammal, an effective amount of a compound of paragraph 48, or a pharmaceutically acceptable salt thereof.
51. A method of treating a mammal having a disease, disorder, or condition responsive to increasing NAMPT activity within the mammal, wherein said method comprises administering, to said mammal, a therapeutically effective amount of a compound of paragraph 48, or a pharmaceutically acceptable salt thereof.
52. The method of paragraph 51, wherein said mammal has a traumatic nerve injury, a neuropathy, a neurodegenerative condition, a central demyelinating disorder, a peripheral demyelinating disorder, a primarily inflammatory neuropathy, glaucoma, an ischemic injury, a retinal or optic nerve ischemia, a stroke, age-related vision or hearing loss, hepatosteatosis, an insulin resistance syndrome, obesity, sarcopenia, a disorder of inflammation or auto-immunity, skin aging, a cardiovascular disease, heart failure, an acute or chronic kidney injury, or infertility.

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A method for increasing NAMPT activity within a mammal, wherein said method comprises administering, to said mammal, an effective amount of a compound of Formula (I): 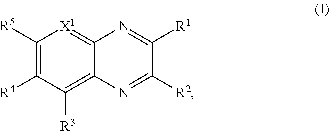
or a pharmaceutically acceptable salt thereof, wherein:
X¹ is selected from N and CR⁶;
R³, R⁴, R⁵, and R⁶ are each independently selected from H, Cy¹, halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, C(NR^{e1})NR^{c1}R^{d1}, C(NR^{e1})NR^{c1}OR^{a1}, NR^{c1}C(O)NR^{c1}R^{d1}, NR^{c1}C(S)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from R⁷;
each R⁷ is independently selected from Cy¹, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
each Cy¹ is independently selected from C₆₋₁₀ aryl, C₃₋₁₀ cycloalkyl, 5-10 membered heteroaryl, and 4-10 membered heterocycloalkyl, each of which is optionally substituted with 1, 2, 3, 4, or 5 substituents independently selected from R^{Cy1};
each R^{Cy1} is independently selected from halo, CN, NO₂, C₁₋₆ alkyl, C₁₋₆ haloalkyl, C₂₋₆ alkenyl, C₂₋₆ alkynyl, C₃₋₁₀ cycloalkyl, C₆₋₁₀ aryl, 5-10 membered heteroaryl, 4-10 membered heterocycloalkyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}, wherein said C₁₋₆ alkyl, C₂₋₆ alkenyl, C₂₋₆ alkynyl, C₃₋₁₀ cycloalkyl, C₆₋₁₀ aryl, 5-10 membered heteroaryl, and 4-10 membered heterocycloalkyl are each optionally substituted with 1, 2, or 3 substituents independently selected from R⁸;
each R⁸ is independently selected from C₁₋₆ alkyl, C₂₋₆ alkenyl, C₂₋₆ alkynyl, C₃₋₁₀ cycloalkyl, C₆₋₁₀ aryl, 5-10 membered heteroaryl, 4-10 membered heterocycloalkyl, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}, wherein said C₁₋₆ alkyl, C₂₋₆ alkenyl, C₂₋₆ alkynyl, C₃₋₁₀ cycloalkyl, C₆₋₁₀ aryl, 5-10 membered heteroaryl, and 4-10 membered heterocycloalkyl are each optionally substituted with 1, 2, 3, 4, or 5 substituents independently selected from R^{g};
R¹ and R² are each independently selected from R⁹ and S(O)₂R⁹;
each R⁹ is independently selected from C₁₋₆ haloalkyl, C₆₋₁₀ aryl, 5-10 membered heteroaryl, and 4-10 membered heterocycloalkyl, each of which is optionally substituted with 1, 2, 3, 4, or 5 substituents independently selected from R¹⁰;
each R¹⁰ is independently selected from halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from R¹¹;
each R¹¹ is independently selected from CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
each R^{e1} is selected from H, OR^{a1}, NR^{c1}R^{d1}, and C₁₋₄ haloalkyl;
each R^{a1}, R^{b1}, R^{c1}, and R^{d1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, 3, 4, or 5 substituents independently selected from R^{g};
or any R^{c1} and R^{d1} together with the N atom to which they are attached form a 4-7 membered heterocycloalkyl, which is optionally substituted with 1, 2, or 3 substituents independently selected from R^{g}; and
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkenylcarbonyl, C₁₋₆ alkynylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino, and any C₁₋₆ alkyl, C₁₋₆ alkoxy, C₆₋₁₀ aryl, C₃₋₁₀ cycloalkyl, 5-10 membered heteroaryl, or 4-10 membered heterocycloalkyl of R^{g} is optionally substituted with 1, 2, or 3 substituents independently selected from OH, NO₂, CN, halo, C₁₋₆ alkyl, C₁₋₄ haloalkyl, C₁₋₆ alkoxy, and C₁₋₆ haloalkoxy.

2. The method of claim 1, wherein the compound of Formula (I) has formula: 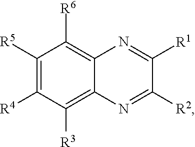
or a pharmaceutically acceptable salt thereof.

3. The method of claim 2, wherein:
R³, R⁵, and R⁶ are each independently selected from H, halo, C₁₋₆ alkyl, C₁₋₆ haloalkyl, OR^{a1}, C(O)OR^{a1}, and NR^{c1}R^{d1}, wherein said C₁₋₆ alkyl is optionally substituted with a substituent selected from OR^{a1}, C(O)R^{b1}, C(O)NR^{c1}R^{d1}, C(O)OR^{a1}, NR^{c1}R^{d1}, S(O)₂R^{b1}, and S(O)₂NR^{c1}R^{d1}; and
R⁴ is selected from Cy¹, C(O)NR^{c1}R^{d1}, C(O)OR^{a1}, NR^{c1}R^{d1}, NR^{c1}C(O)NR^{c1}R^{d1}, NR^{o1}C(S)NR^{c1}R^{d1}, and S(O)₂NR^{c1}R^{d1}.

4. The method of claim 3, wherein:
R³, R⁵, and R⁶ are each independently selected from H, halo, C₁₋₆ alkyl, C₁₋₆ haloalkyl, C₁₋₆ alkoxy, C₁₋₆ haloalkoxy; and
R⁴ is selected from Cy¹, C(O)NR^{c1}R^{d1} and C(O)OR^{a1}.

5. The method of claim 1, wherein the compound of Formula (I) has formula: 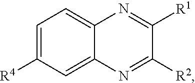
or a pharmaceutically acceptable salt thereof.

6. The method of claim 1, wherein the compound of Formula (I) has formula: 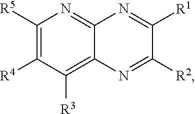
or a pharmaceutically acceptable salt thereof.

7. The method of claim 6, wherein:
R³ and R⁵ are each independently selected from H, halo, C₁₋₆ alkyl, C₁₋₆ haloalkyl, OR^{a1}, C(O)OR^{a1}, and NR^{c1}R^{d1}, wherein said C₁₋₆ alkyl is optionally substituted with a substituent selected from OR^{a1}, C(O)R^{b1}, C(O)NR^{c1}R^{d1}, C(O)OR^{a1}, NR^{c1}R^{d1}, S(O)₂R^{b1}, and S(O)₂NR^{c1}R^{d1}; and
R⁴ is selected from Cy¹, C(O)NR^{c1}R^{d1}, C(O)OR^{a1}, NR^{c1}R^{d1}, NR^{c1}C(O)NR^{c1}R^{d1}, NR^{c1}C(S)NR^{c1}R^{d1}, and S(O)₂NR^{c1}R^{d1}.

8. The method of claim 7, wherein:
R³ and R⁵ are each independently selected from H, halo, C₁₋₆ alkyl, C₁₋₆ haloalkyl, C₁₋₆ alkoxy, C₁₋₆ haloalkoxy; and
R⁴ is selected from Cy¹, C(O)NR^{c1}R^{d1}, and C(O)OR^{a1}.

9. The method of claim 1, wherein the compound of Formula (I) has formula: 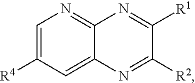
or a pharmaceutically acceptable salt thereof.

10. The method of claim 1, wherein the compound of Formula (I) has formula: 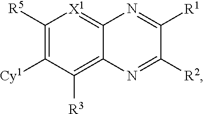
or a pharmaceutically acceptable salt thereof.

11. The method of claim 1, wherein the compound of Formula (I) has formula: 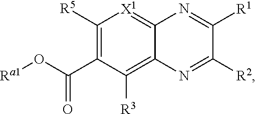
or a pharmaceutically acceptable salt thereof.

12. The method of claim 1, wherein the compound of Formula (I) has formula: 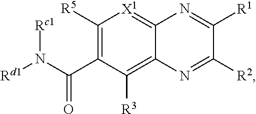
or a pharmaceutically acceptable salt thereof.

13. The method of claim 12, wherein the compound of Formula (I) has formula: 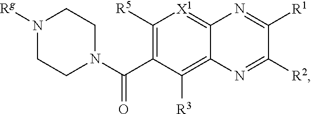
or a pharmaceutically acceptable salt thereof.

14. The method of claim 1, wherein R¹ and R² are each independently selected from C₁₋₆ haloalkyl, C₆₋₁₀ aryl, and 5-10 membered heteroaryl, wherein said C₆₋₁₀ aryl and 5-10 membered heteroaryl are each optionally substituted with 1 or 2 independently selected from R¹⁰.

15. The method of claim 14, wherein R¹ and R² are each independently a C₆₋₁₀ aryl, optionally substituted with 1 or 2 independently selected from R¹⁰.

16. The method of claim 14, wherein:
R¹ is 5-10 membered heteroaryl, optionally substituted with 1 or 2 independently selected from R¹⁰; and
R² is C₆₋₁₀ aryl, optionally substituted with 1 or 2 independently selected from R¹⁰; or
R¹ is C₆₋₁₀ aryl, optionally substituted with 1 or 2 independently selected from R¹⁰; and
R² is 5-10 membered heteroaryl, optionally substituted with 1 or 2 independently selected from R¹⁰; or
R¹ is C₁₋₆ haloalkyl; and
R² is C₆₋₁₀ aryl, optionally substituted with 1 or 2 independently selected from R¹⁰; or
R¹ is C₁₋₆ haloalkyl; and
R² is 5-10 membered heteroaryl, optionally substituted with 1 or 2 independently selected from R¹⁰; or
R¹ is C₆₋₁₀ aryl, optionally substituted with 1 or 2 independently selected from R¹⁰; and
R² is C₁₋₆ haloalkyl.

17-20. (canceled)

21. The method of claim 1, wherein:
R³, R⁵, and R⁶ are each independently selected from H, halo, C₁₋₆ alkyl, C₁₋₆ haloalkyl, OR^{a1}, C(O)OR^{a1}, and NR^{c1}R^{d1}, wherein said C₁₋₆ alkyl is optionally substituted with a substituent selected from OR^{a1}, C(O)R^{b1}, C(O)NR^{c1}R^{d1}, C(O)OR^{a1}, NR^{c1}R^{d1}, S(O)₂R^{b1}, and S(O)₂NR^{c1}R^{d1};
R⁴ is selected from Cy¹, C(O)NR^{c1}R^{d1}, C(O)OR^{a1}, NR^{c1}R^{d1}, NR^{c1}C(O)NR^{c1}R^{d1}, NR^{c1}C(S)NR^{c1}R^{d1}, and S(O)₂NR^{c1}R^{d1};
R^{c1} and R^{d1} are each independently selected from H, C₁₋₆ alkyl, C₁₋₄ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with a substituent selected from OH, thio, C₁₋₆ alkoxy, C₁₋₆ thialkoxy, C₁₋₆ haloalkoxy, amino, C₁₋₆ alkylamino, and di(C₁₋₆ alkyl)amino; or
R^{c1} and R^{d1} together with the N atom to which they are attached form a 4-7 membered heterocycloalkyl, which is optionally substituted with 1 or 2 substituents independently selected from R^{g};
R^{a1} is selected from H, C₁₋₆ alkyl, C₁₋₄ haloalkyl, C₂₋₆ alkenyl, and C₂₋₆ alkynyl, wherein said C₁₋₆ alkyl, C₂₋₆ alkenyl, and C₂₋₆ alkynyl are each optionally substituted with a substituent selected from OH, thio, C₁₋₆ alkoxy, C₁₋₆ thialkoxy, C₁₋₆ haloalkoxy, amino, C₁₋₆ alkylamino, and di(C₁₋₆ alkyl)amino;
Cy¹ is 5-10 membered heteroaryl, which is optionally substituted with 1 or 2 independently selected R^{Cy1};
R^{Cy1} is selected from C₁₋₆ alkyl, C₁₋₆ haloalkyl, C₂₋₆ alkenyl, and C₂₋₆ alkynyl, each of which is optionally substituted with 1 or 2 independently selected R⁸;
R⁸ is selected from OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
R¹ and R² are each independently selected from C₁₋₆ haloalkyl, C₆₋₁₀ aryl, and 5-10 membered heteroaryl, wherein said C₆₋₁₀ aryl and 5-10 membered heteroaryl are each optionally substituted with 1 or 2 independently selected from R¹⁰; and
R¹⁰ is selected from halo, CN, C(O)NR^{c1}R^{d1}, C(O)OR^{a1}, NR^{c1}R^{d1}, NR^{o1}C(O)R^{b1}, NR^{o1}S(O)₂R^{b1}, S(O)₂R^{b1}, and S(O)₂NR^{c1}R^{d1}.

22. The method of claim 1, wherein:
R³, R⁵, and R⁶ are each independently selected from H, halo, C₁₋₆ alkyl, C₁₋₆ haloalkyl, C₁₋₆ alkoxy, and C₁₋₆ haloalkoxy; and
R⁴ is selected from Cy¹, C(O)NR^{c1}R^{d1}, and C(O)OR^{a1};
R^{c1} and R^{d1} are each independently selected from H, C₁₋₆ alkyl, and C₂₋₆ alkynyl, wherein said C₁₋₆ alkyl is optionally substituted with a substituent selected from OH, C₁₋₆ alkoxy, and di(C₁₋₆ alkyl)amino; or
R^{c1} and R^{d1} together with the N atom to which they are attached form piperazinyl, optionally substituted with 1 or 2 substituents independently selected from R^{g};
R^{a1} is selected from H and C₁₋₆ alkyl;
Cy¹ is selected from 1,2,4-triazolyl, tetrazolyl, oxazolyl, isoxazolyl, thiophenyl, indolyl, pyrimidinyl, pyrrolopyridinyl, benzoxadiazolyl, 1,3,4-oxadiazolyl, 1,2,3-triazolyl, 1,2,4-oxadiazolyl, thiazolyl, pyridinyl, benzoxazinyl, pyrazolyl, and indazolyl, each of which is optionally substituted with 1 or 2 substituents independently selected from R^{c311};
R^{Cy1} is C₁₋₆ alkyl, optionally substituted with R⁸;
R⁸ is selected from OH, thio, C₁₋₆ alkoxy, C₁₋₆ thialkoxy, C₁₋₆ haloalkoxy, amino, C₁₋₆ alkylamino, and di(C₁₋₆ alkyl)amino;
R¹ and R² are each independently selected from C₁₋₆ haloalkyl, C₆₋₁₀ aryl, and 5-10 membered heteroaryl, wherein said C₆₋₁₀ aryl and 5-10 membered heteroaryl are each optionally substituted with 1 or 2 independently selected from R¹⁰; and
R¹⁰ is selected from halo, CN, NR^{c1}R^{d1}, NR^{c1}C(O)R^{b1}, and S(O)₂R^{b1}.

23. The method of claim 1, wherein the compound of Formula (I) is selected from any one of the following compounds: 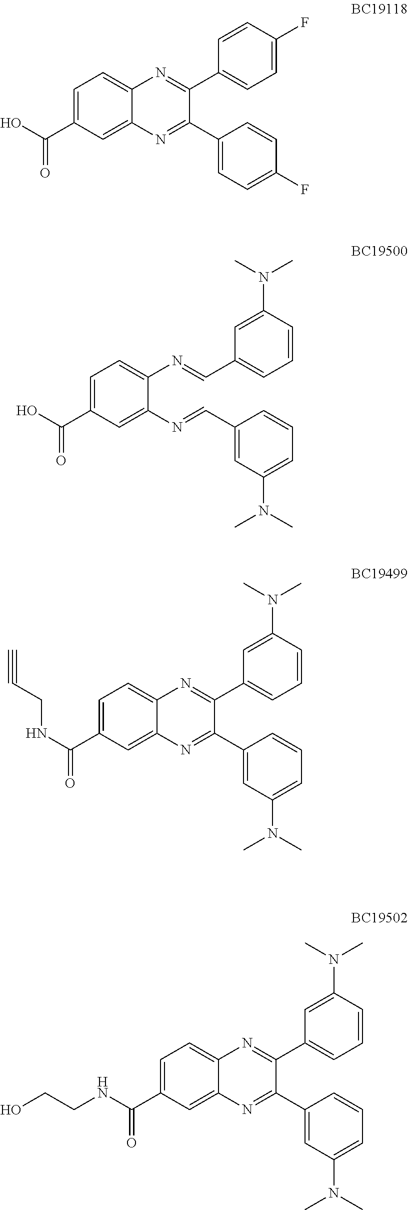 [SMILES: O=C(O)c1ccc2nc(-c3ccc(F)cc3)c(-c3ccc(F)cc3)nc2c1 ; CN(C)c1cccc(/C=N/c2ccc(C(=O)O)cc2/N=C/c2cccc(N(C)C)c2)c1 ; C#CCNC(=O)c1ccc2nc(-c3cccc(N(C)C)c3)c(-c3cccc(N(C)C)c3)nc2c1 ; CN(C)c1cccc(-c2nc3ccc(C(=O)NCCO)cc3nc2-c2cccc(N(C)C)c2)c1] 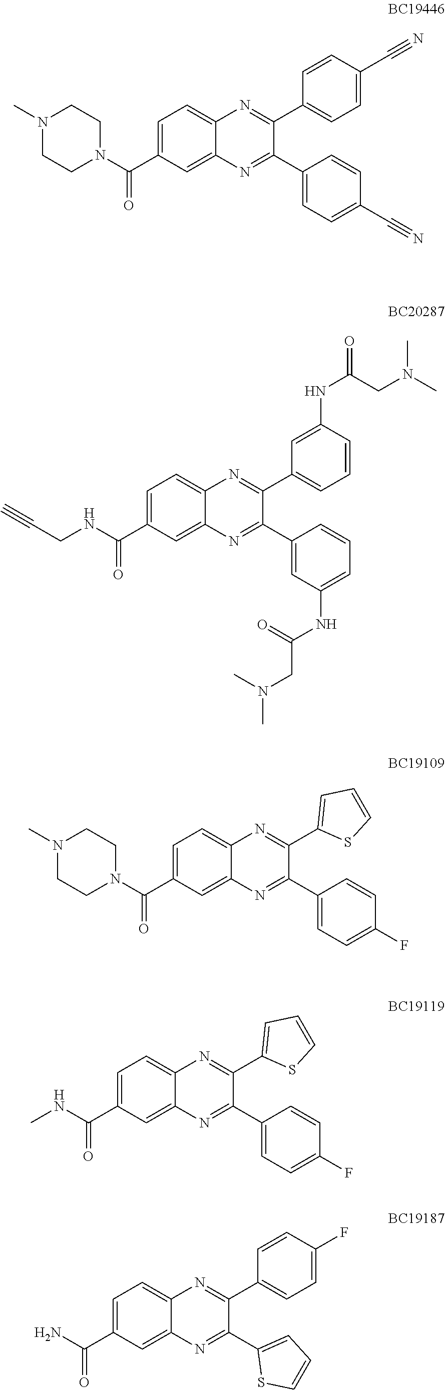 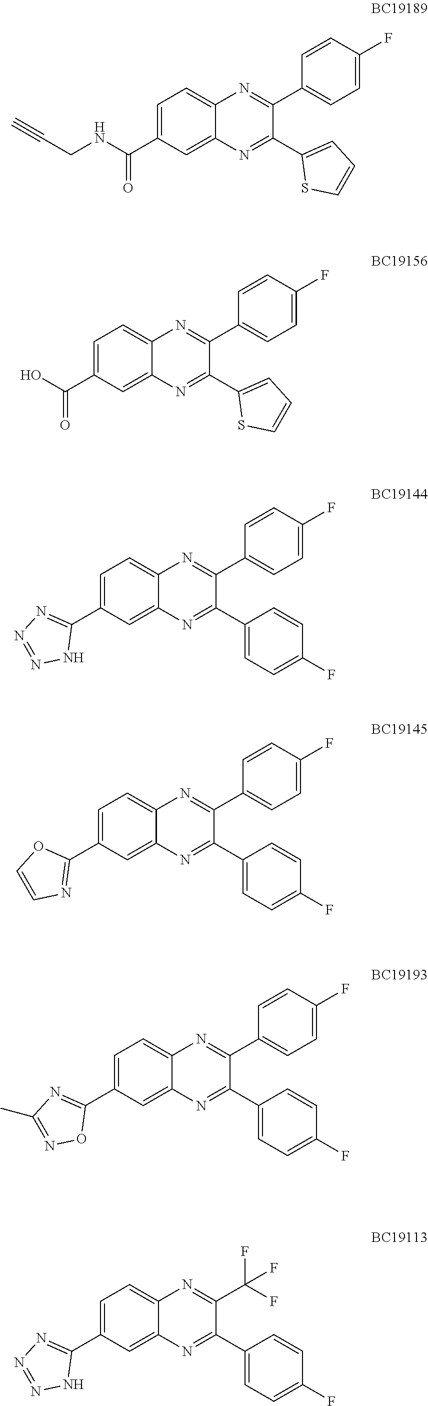 [SMILES: C#CCNC(=O)c1ccc2nc(-c3ccc(F)cc3)c(-c3cccs3)nc2c1 ; O=C(O)c1ccc2nc(-c3ccc(F)cc3)c(-c3cccs3)nc2c1 ; Fc1ccc(-c2nc3ccc(-c4nnn[nH]4)cc3nc2-c2ccc(F)cc2)cc1 ; Fc1ccc(-c2nc3ccc(-c4ncco4)cc3nc2-c2ccc(F)cc2)cc1 ; Cc1noc(-c2ccc3nc(-c4ccc(F)cc4)c(-c4ccc(F)cc4)nc3c2)n1 ; Fc1ccc(-c2nc3cc(-c4nnn[nH]4)ccc3nc2C(F)(F)F)cc1] 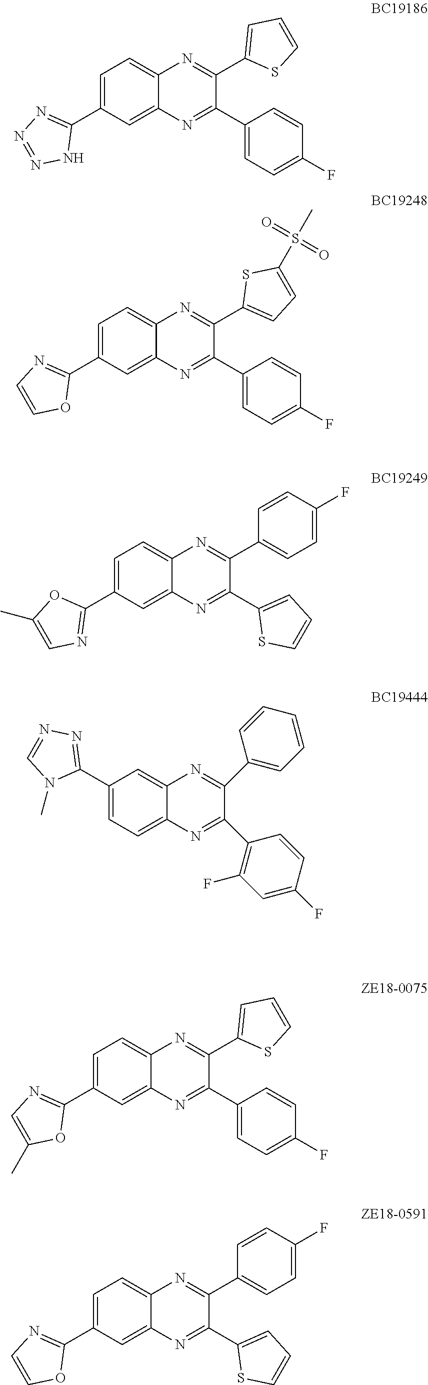 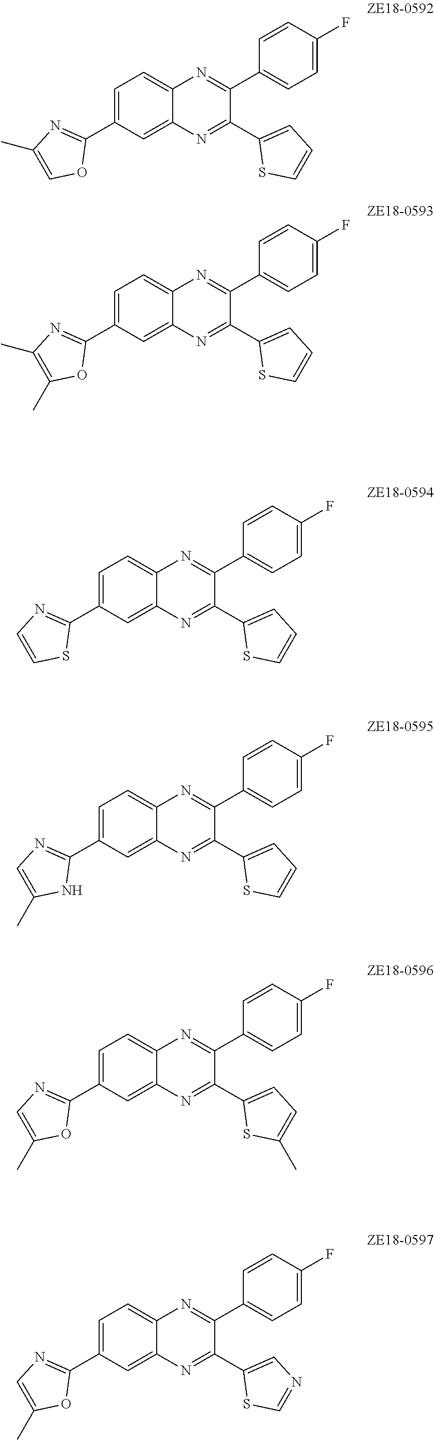 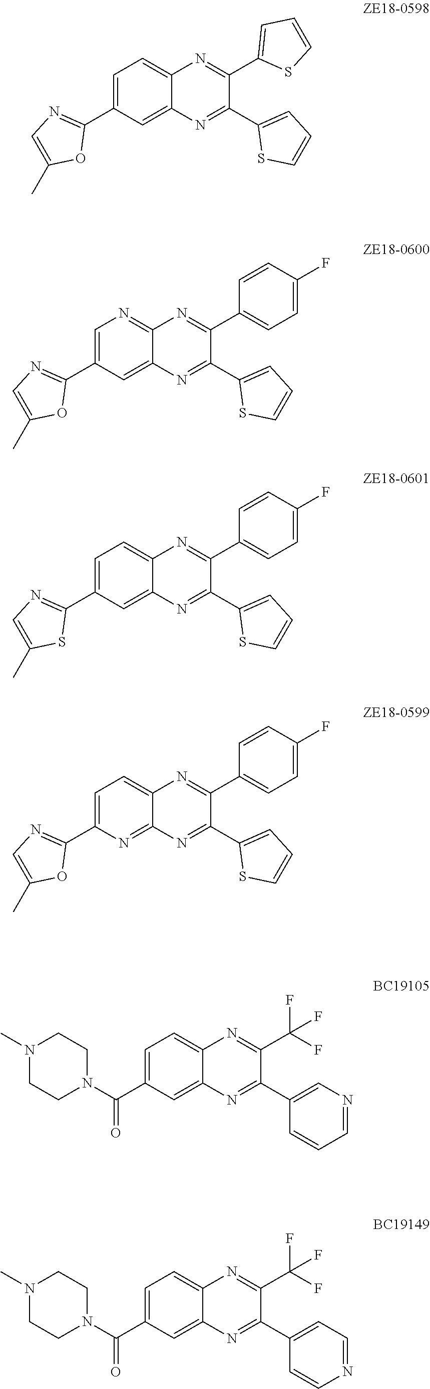 [SMILES: Cc1cnc(-c2ccc3nc(-c4cccs4)c(-c4cccs4)nc3c2)o1 ; Cc1cnc(-c2cnc3nc(-c4ccc(F)cc4)c(-c4cccs4)nc3c2)o1 ; Cc1cnc(-c2ccc3nc(-c4ccc(F)cc4)c(-c4cccs4)nc3c2)s1 ; Cc1cnc(-c2ccc3nc(-c4ccc(F)cc4)c(-c4cccs4)nc3n2)o1 ; CN1CCN(C(=O)c2ccc3nc(C(F)(F)F)c(-c4cccnc4)nc3c2)CC1 ; CN1CCN(C(=O)c2ccc3nc(C(F)(F)F)c(-c4ccncc4)nc3c2)CC1] 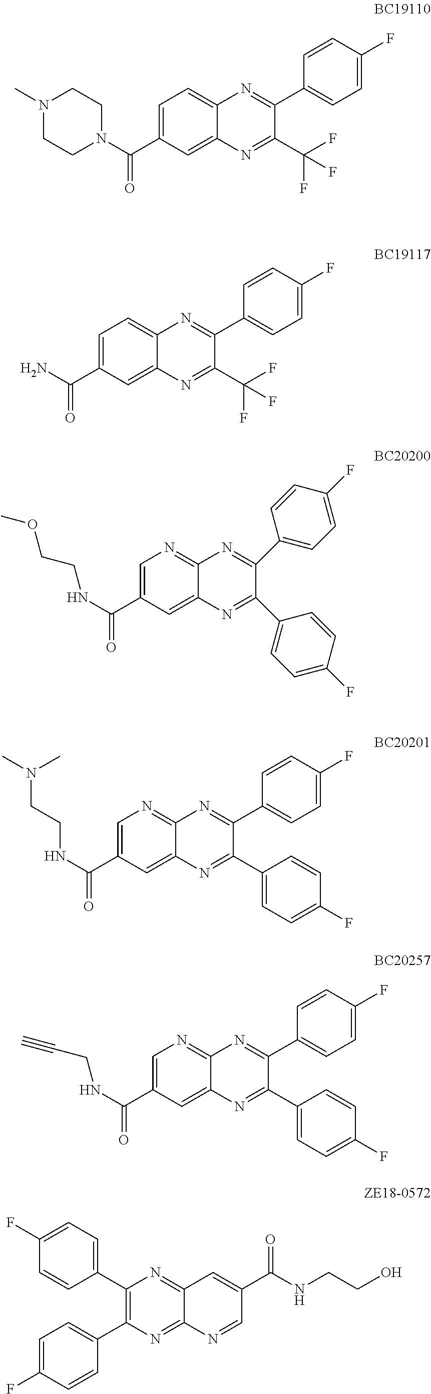 [SMILES: CN1CCN(C(=O)c2ccc3nc(-c4ccc(F)cc4)c(C(F)(F)F)nc3c2)CC1 ; NC(=O)c1ccc2nc(-c3ccc(F)cc3)c(C(F)(F)F)nc2c1 ; COCCNC(=O)c1cnc2nc(-c3ccc(F)cc3)c(-c3ccc(F)cc3)nc2c1 ; CN(C)CCNC(=O)c1cnc2nc(-c3ccc(F)cc3)c(-c3ccc(F)cc3)nc2c1 ; C#CCNC(=O)c1cnc2nc(-c3ccc(F)cc3)c(-c3ccc(F)cc3)nc2c1 ; O=C(NCCO)c1cnc2nc(-c3ccc(F)cc3)c(-c3ccc(F)cc3)nc2c1] 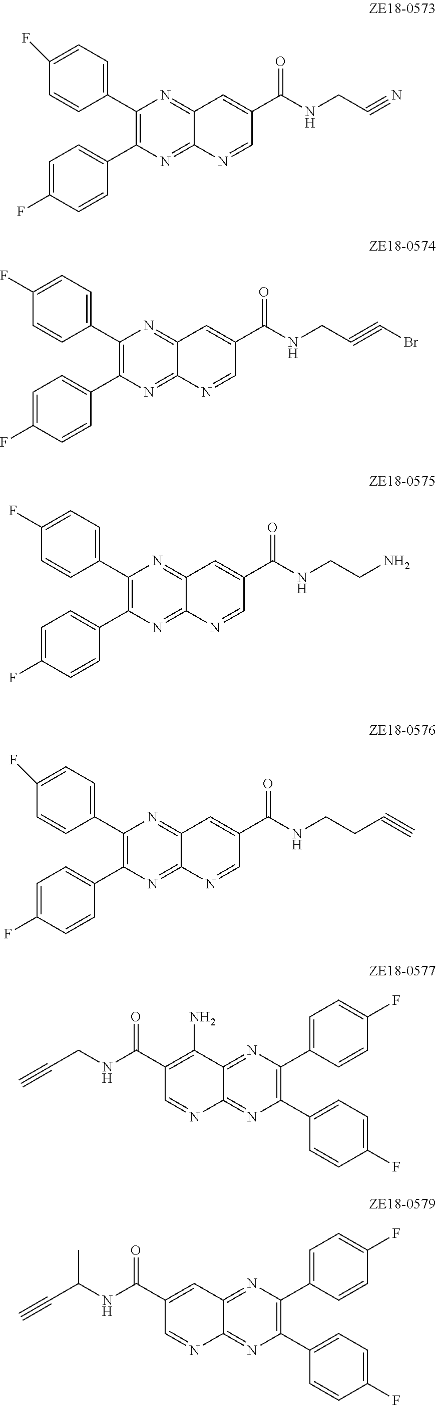 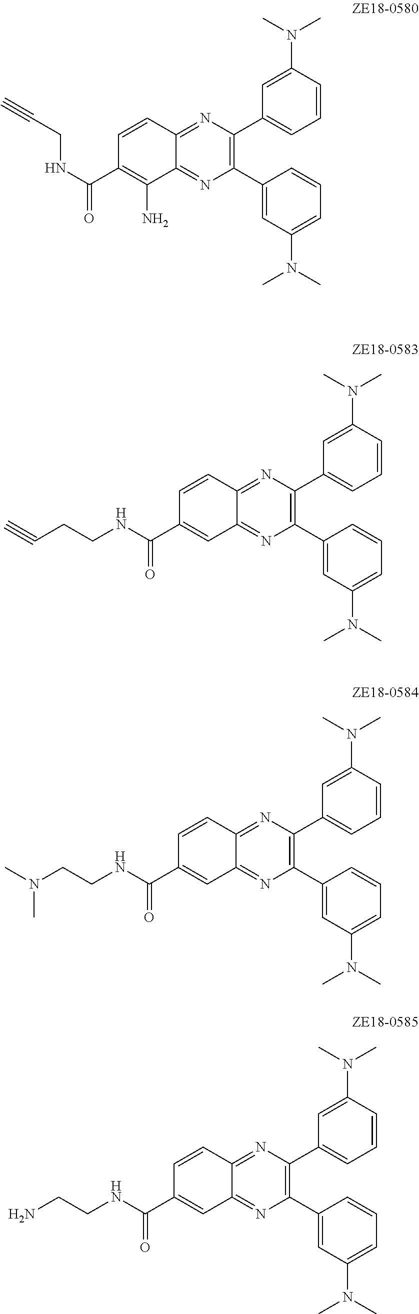 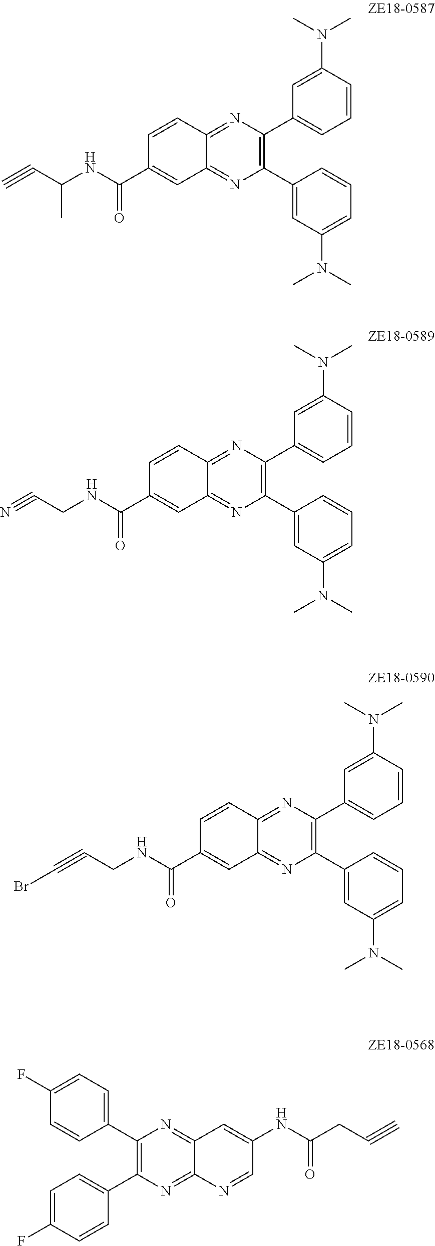 [SMILES: C#CC(C)NC(=O)c1ccc2nc(-c3cccc(N(C)C)c3)c(-c3cccc(N(C)C)c3)nc2c1 ; CN(C)c1cccc(-c2nc3ccc(C(=O)NCC#N)cc3nc2-c2cccc(N(C)C)c2)c1 ; CN(C)c1cccc(-c2nc3ccc(C(=O)NCC#CBr)cc3nc2-c2cccc(N(C)C)c2)c1 ; C#CCC(=O)Nc1cnc2nc(-c3ccc(F)cc3)c(-c3ccc(F)cc3)nc2c1] 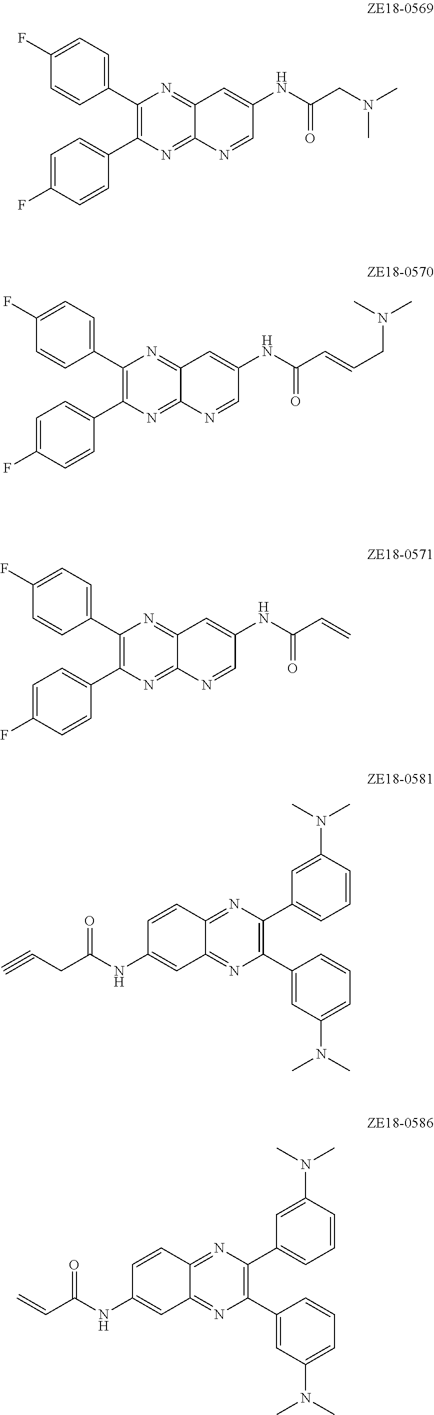 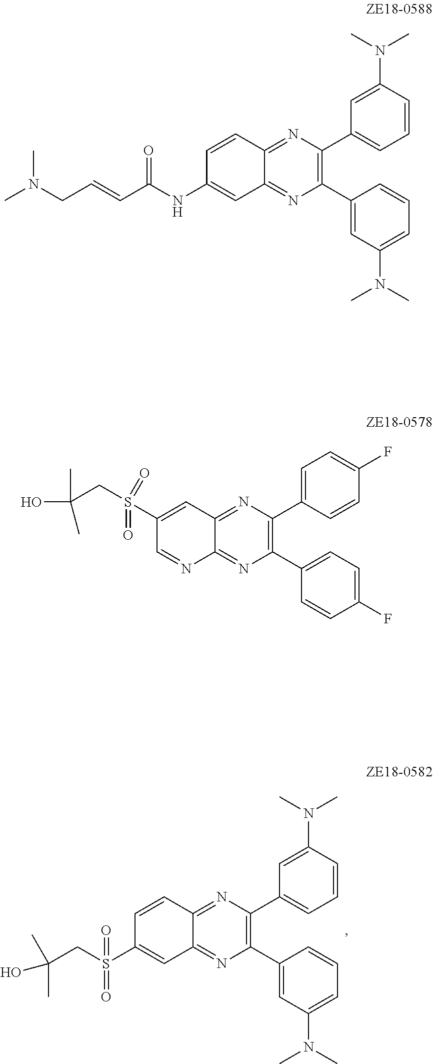
or a pharmaceutically acceptable salt thereof.

24. The method of claim 1, wherein said method is a method for treating a disease or condition responsive to increasing NAMPT activity within a mammal selected from a traumatic nerve injury, a neuropathy, a neurodegenerative condition, a central demyelinating disorder, a peripheral demyelinating disorder, a primarily inflammatory neuropathy, glaucoma, an ischemic injury, a retinal or optic nerve ischemia, a stroke, age-related vision or hearing loss, hepatosteatosis, an insulin resistance syndrome, obesity, sarcopenia, a disorder of inflammation or auto-immunity, skin aging, a cardiovascular disease, heart failure, an acute or chronic kidney injury, and infertility.

25. (canceled)
